(19) United States
(12) Patent Application Publication
(10) Pub. No.: US 2026/0263603 A1
(43) Pub. Date: Sep. 10, 2026

(54) PRODRUGS, PRODRUG COMPOSITIONS AND RELATED METHODS

(71) Applicant: Praesidia Biotherapeutics Inc., Arlington, MA (US)

(72) Inventors: Ahmed Masud Aman, Oakville, Ontario (CA); Pradip Kumar Majumder, Chestnut Hill, MA (US); Salma Shirin, Oakville, Ontario (CA); Sohang Chatterjee, Lexington, MA (US)

(21) Appl. No.: 19/167,739

(22) PCT Filed: Mar. 22, 2024
(86) PCT No.: PCT/US2024/021138

§ 371 Date: Sep. 22, 2025

Related U.S. Application Data
(60) Provisional application No. 63/491,873, filed on Mar. 23, 2023.

(51) Int. Cl. A61K 47/54; A61K 31/573; A61K 31/704

(52) U.S. Cl. CPC A61K 47/545; A61K 31/573; A61K 31/704

(57) ABSTRACT

Embodiments of the present invention generally relate to prodrugs, and more particularly to prodrugs capable of targeting brain-related diseases.

### CROSS REFERENCE TO RELATED APPLICATION

This application claims a benefit of, and priority to, U.S. Provisional Patent Application No. 63/491,873, filed Mar. 23, 2023, the disclosure of which is incorporated by reference in its entirety.

### TECHNICAL FIELD

Embodiments of the present invention generally relate to prodrugs, and more particularly to prodrugs capable of targeting brain-related diseases.

### BACKGROUND

The blood-brain barrier (BBB) is a system-wide membrane barrier that prevents the brain uptake of circulating drugs, protein therapeutics, RNAi drugs, and gene medicines. Drugs or genes can be delivered to the human brain for the treatment of serious brain disease either (a) by injecting the drug or gene directly into the brain, thus bypassing the BBB, or (b) by injecting the drug or gene into the bloodstream so that the drug or gene enters the brain via the transvascular route across the BBB. Intra-cerebral administration of the drug is highly invasive and not very effective. The transvascular route is non-invasive and can potentially allow for wider distribution of the drug to the target cells in the brain. However, this latter approach requires the ability to undergo transport across the BBB, which has been a difficult barrier to traverse safely.

The development of prodrugs has become one of the strategies to improve the physicochemical, pharmacokinetic, and/or pharmacodynamic properties of pharmaceutically active agents, and thereby enhance their efficacy and/or reduce their side effects. For example, prodrugs provide possibilities to overcome various barriers to drug formulation and delivery such as poor aqueous solubility, physical and/or chemical instability, insufficient absorption, rapid pre-systemic metabolism, inadequate brain penetration, toxicity, and/or local irritation. Prodrugs may also be effective in improving BBB transport, drug targeting and duration of action. Prodrugs are typically inactive derivatives of a drug molecule that require chemical or enzymatic biotransformation to release the active parent drug in the body. Therefore, prodrugs need to be efficiently converted to the parent drugs to reach pronounced efficacy as soon as the drug target has been reached.

Thus, there is a need for new prodrug chemistries that provide for the effective transport of the drug across the BBB. Moreover, there is a need for new prodrug chemistries that provide for specific targeting to target cells and/or target sites in the brain while minimizing toxicity.

### SUMMARY

The following summary is illustrative only and is not intended to be in any way limiting. In addition to the illustrative aspects, example embodiments, and features described, further aspects, example embodiments, and features will become apparent by reference to the following detailed description.

In some aspects of the present invention, a compound having a structure of formula (I), or a pharmaceutically acceptable salt thereof is presented. Formula (I) is:

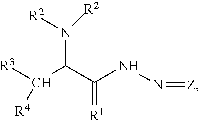

wherein Z is a therapeutic moiety;
R¹ is O or S;
R² is independently at each occurrence hydrogen or a C₁-C₃ alkyl group;
R³ is hydrogen or a C₁-C₃ alkyl group; and
R⁴ is a C₁-C₆ alkyl group, a C₆-C₁₀ aryl group, or a C₅-C₁₀ heteroaryl group.

In some aspects of the present invention, a pharmaceutical composition is presented. The pharmaceutical composition includes a compound of formula (I) or a pharmaceutically acceptable salt, solvate, hydrate, polymorph, or co-crystal thereof, and a pharmaceutically carrier, diluent, or excipient.

In some aspects of the present invention, a method of treating a brain disease is presented. The method includes administering to a patient an effective amount of a pharmaceutical composition including a compound of formula (I) or a pharmaceutically acceptable salt, solvate, hydrate, polymorph, or co-crystal thereof.

### DETAILED DESCRIPTION OF EXAMPLE EMBODIMENTS

Various example embodiments will now be described more fully with reference to the accompanying drawings in which only some example embodiments are shown. Specific structural and functional details disclosed herein are merely representative for purposes of describing example embodiments. Example embodiments, however, may be embodied in many alternate forms and should not be construed as limited to only the example embodiments set forth herein. On the contrary, example embodiments are to cover all modifications, equivalents, and alternatives thereof.

The terminology used herein is for the purpose of describing particular example embodiments only and is not intended to be limiting. Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

To more clearly and concisely describe and point out the subject matter of the claimed invention, the following definitions are provided for specific terms that are used in the following description and the claims appended hereto. Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Unless otherwise stated, structures depicted herein are also meant to include compounds that differ only in the presence of one or more isotopically enriched atoms. For example, compounds having the present structures including the replacement of hydrogen by deuterium or tritium, the replacement of a carbon by a 13C- or 14C-enriched carbon, or the replacement of a fluorine by 18F-enriched fluorine, are within the scope of this invention. Such compounds are useful, for example, as analytical tools, as probes in biological assays, or as therapeutic agents in accordance with the present invention.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high-pressure liquid chromatography (HPLC) and the formation and crystallization of chiral salts or prepared by asymmetric syntheses.

As used herein the terms “L isomer” and “D isomer” refer to the enantiomers of amino acids. Amino acids are found naturally in two forms (L and D enantiomers), except for glycine which does not have a chiral center. L and D enantiomers have different orientation of the four substituents attached to the α-carbon and also known as non-superimposed mirror images.

Ass used herein the terms “syn isomer” and “anti isomer” refer to diastereomers of the compounds of the present invention. Diastereomers are defined as non-mirror image, non-identical stereoisomers. Hence, they occur when two or more stereoisomers of a compound have different configurations at one or more (but not all) of the equivalent (related) stereocenters and are not mirror images of each other. The prefixes syn and anti are used to denote geometrical isomerism. Ther term “syn isomer” indicates that the two functional groups are present on the same side of the double bond (e.g., N═C bond). Ther term “anti isomer” indicates that the two functional groups are present on the opposite sides of the double bond (e.g., N═C bond).

As used herein, the term “alkyl group” refers to a saturated monovalent group consisting of a linear or branched array of atoms that is not cyclic. Alkyl groups are defined to include at least one carbon atom and are represented by formula CₙH₂ₙ₊₁. The array of atoms included in the alkyl group may be composed exclusively of carbon and hydrogen. By way of example, the term “C₁-C₁₀ alkyl group” contains at least one but no more than 10 carbon atoms. A methyl group (i.e. CH₃—) is an example of a monovalent C₁ alkyl group. A decyl group (i.e., CH₃(CH₂)₉) is an example of a monovalent C₁₀ alkyl group. Non-limiting examples of alkyl groups include methyl, ethyl, n-propyl, isopropyl, n-butyl, t-butyl, isobutyl, sec-butyl, n-pentyl, n-hexyl, n-heptyl, n-octyl, and the like.

As used herein the term “aryl group” refers to a monovalent aromatic hydrocarbon radical derived by the removal of one hydrogen atom from a single carbon atom of a parent aromatic ring system. Aryl group encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene.

The term “aryl group “also encompasses multiple ring systems having at least one carbocyclic aromatic ring fused to at least one carbocyclic aromatic ring, cycloalkyl ring, or heterocycloalkyl ring. For example, aryl group includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can include from 5 to 20 carbon atoms, and in certain embodiments, from 5 to 12 carbon atoms.

As used herein, the term “heteroaryl group” refers to a monovalent heteroaromatic radical derived by the removal of one hydrogen atom from a single atom of a parent heteroaromatic ring system. Heteroaryl group encompasses multiple ring systems having at least one aromatic ring fused to at least one other ring, which can be aromatic or non-aromatic in which at least one ring atom is a heteroatom. Heteroaryl group encompasses 5- to 12-membered aromatic, monocyclic rings (such as 5- to 7-membered rings) containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heteroaromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one.

Examples of heteroaryl groups include, but are not limited to, groups derived from acridine, arsindole, carbazole, beta-carboline, chromane, chromene, cinnoline, furan, furazan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

As used herein, the term “amino acid” refers to both naturally occurring and non-naturally occurring amino acids. Therefore, the term “amino acid” includes naturally occurring proteogenic L-amino acids; D-amino acids; chemically modified amino acids such as amino acid variants and derivatives; naturally occurring non-proteogenic amino acids such as norleucine, β-alanine, omithine, etc.; and chemically synthesized compounds having properties known in the art to be characteristic of amino acids.

Some of the standard naturally occurring nonpolar (hydrophobic) amino acids include alanine (Ala), leucine (Leu), isoleucine (Ile), valine (Val), proline (Pro), phenylalanine (Phe), tryptophan (Trp) and methionine (Met). The polar neutral amino acids include glycine (Gly), serine (Ser), threonine (Thr), cysteine (Cys), tyrosine (Tyr), asparagine (Asn), and glutamine (Gln). The positively charged (basic) amino acids include arginine (Arg), lysine (Lys), and histidine (His). The negatively charged (acidic) amino acids include aspartic acid (Asp) and glutamic acid (Glu). The non-standard amino acids may be formed, for example, in the body by posttranslational modification. Some examples of such naturally occurring non-standard amino acids include selenocysteine and pyrolysine. Other non-limiting examples of amino acids include α-methyl amino acids (e.g., α-methyl alanine), β-amino acids, histidine-like amino acids (e.g., 2-amino-histidine, β-hydroxy-histidine, homohistidine, α-fluoromethyl-histidine, and α-methyl-histidine), amino acids having an extra methylene in the side chain (“homo” amino acids) and amino acids in which a carboxylic acid functional group in the side chain is replaced with a sulfonic acid group (e.g., cysteic acid).

The term “amino acid” as used herein encompasses amino acids wherein one or more amino groups in the amino acid compounds may be further substituted with an alkyl group to form a monoalkyl amino acid or a dialkyl amino acid. For example, N,N-dimethyl-L-phenylalanine is an example of a dialkyl amino acid and methyl-L-alanine is an example of a monoalkyl amino acid. Further, in the present description, the designation of an amino acid without specifying its stereochemistry is intended to encompass either the L or D form of the amino acid or a racemic mixture thereof.

As used herein the term “cleavable moiety” refers to a moiety having a bond that is cleavable under specified conditions of use, for example, following administration to a patient. The bond may be cleaved by enzymatic or non-enzymatic means. The cleavage may proceed spontaneously, such as via a hydrolysis reaction, or it may be catalyzed or induced by another agent, such as an enzyme, an acid, a base, or a change of or exposure to a physical or environmental parameter, such as temperature, pH, etc. The agent may be endogenous to the conditions of use, such as an enzyme present in the systemic circulation of a patient to which the prodrug is administered or the acidic conditions of the stomach, or the agent may be supplied exogenously. As used herein the term “enzymatically cleavable moiety” refers to a moiety having a bond that is cleavable by enzymatic means. In certain embodiments, the cleavable moiety has a bond that is cleavable by enzymatic means present in the diseased organ, tissue, or cells.

As used herein, the term “specific binding” refers to the specific recognition of one of two different molecules for the other compared to substantially less recognition of other molecules. The molecules may have areas on their surfaces or in cavities giving rise to specific recognition between the two molecules arising from one or more of electrostatic interactions, hydrogen bonding, or hydrophobic interactions. Specific binding examples include, but are not limited to, antibody-antigen interactions, enzyme-substrate interactions, polynucleotide interactions, receptor interactions, and the like. In some embodiments, specific binding refers to the binding of the compounds of the present invention to one or more enzymes located within or in the proximity of the target cells or the target site (such as a target tissue or organ).

As used herein, the term “target,” refers to moieties that are naturally more expressed in a diseased state versus a healthy state, or alternatively, to moieties that are naturally more expressed in the target cells or target site (such as a tissue or an organ). In general, the compounds of the present invention may bind to a target through one or more discrete chemical moieties of the target or a three-dimensional structural component of the target (e.g., 3D structures resulting from peptide folding). The target may include one or more of natural or modified peptides, proteins (e.g., antibodies, affibodies, aptamers, or lectins), nucleic acids (e.g., polynucleotides, DNA, RNA, or aptamers); polysaccharides (e.g., sugars), lipids, enzymes, enzyme substrates, ligands, receptors, antigens, or haptens. In some embodiments, targets may include one or more enzymes present within or in proximity to the target cells or the target site (such as a target tissue or organ). Non-limiting examples of targets include brain amidase, Fatty Acid Amide Hydrolase (FAAH), Kallikrein 6 (KLK6), Acetyl-CoA synthetase 2 (ACSS2), Dipeptidyl-Peptidase 4 (DPP4), and the like.

As used herein, the term “prodrug” refers to a derivative of a drug or a pharmaceutically active agent that is administered in an inactive or less than fully active form and is then converted to its active form within the body. In some embodiments, the transformation releases the parent drug or pharmaceutically active agent. In some embodiments, a bioactive derivative of the parent drug or pharmaceutically active agent is generated.

As used herein, the term “therapeutically effective amount” refers to an amount (of a compound) that is sufficient to provide a therapeutic benefit to a patient in the treatment or management of a disease or disorder, or to delay or minimize one or more symptoms associated with the disease or disorder.

As used herein, the term “pharmaceutically acceptable salt” refers to any salt suitable for administration to a patient. Examples of salts include but are not limited to, acid-derived, base-derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p toluenesulfonic acid, salicylic acid, and the like.

In some embodiments, a compound having a formula (I) or a pharmaceutically acceptable salt thereof is presented.

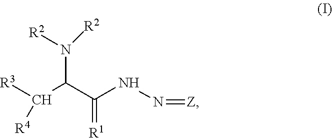

wherein Z is a therapeutic moiety;
R¹ is O or S;
R² is independently at each occurrence hydrogen or a C₁-C₃ alkyl group
R³ is hydrogen or a C₁-C₃ alkyl group; and
R⁴ is a C₁-C₆ alkyl group, a C₆-C₁₀ aryl group, or a C₅-C₁₀ heteroaryl group.

Non-limiting examples of R² include hydrogen, a methyl group, or an ethyl group. Non-limiting examples of R³ include hydrogen or a methyl group. Non-limiting examples of R⁴ include a methyl group, an isopropyl group, a phenyl group, or an indole group.

In some embodiments, the compound is an L and syn isomer of formula (I). In some embodiments, the compound is a D and syn isomer of formula (II). In some embodiments, the compound is an L and anti-isomer of formula (I). In some embodiments, the compound is a D and syn isomer of formula (I).

In some embodiments, a compound having a formula (II) or a pharmaceutically acceptable salt thereof is presented:

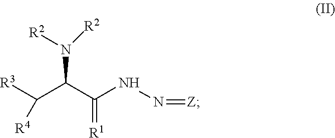

wherein Z is a therapeutic moiety;
R¹ is O or S;
R² is independently at each occurrence hydrogen or a C₁-C₃ alkyl group
R³ is hydrogen or a C₁-C₃ alkyl group; and
R⁴ is a C₁-C₆ alkyl group, a C₆-C₁₀ aryl group, or a C₅-C₁₀ heteroaryl group.

In some embodiments, the compound is a syn isomer of formula (II). In some embodiments, the compound is an anti isomer of formula (II).

In some embodiments, a compound having a formula (III) to (VI) or a pharmaceutically acceptable salt thereof is presented.

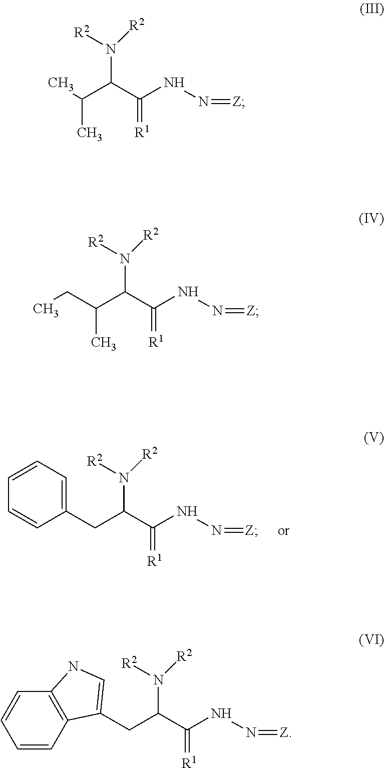

In some embodiments, a compound has a formula (VII) or a pharmaceutically acceptable salt thereof is presented.

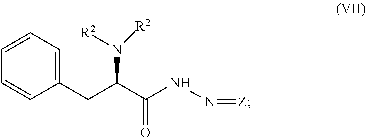

wherein R² is independently at each occurrence hydrogen or a C₁-C₃ alkyl group.

As noted earlier, Z is a therapeutic moiety obtained from a therapeutic agent. Non-limiting examples of therapeutic agents include ACE-inhibitors; anti-anginal drugs; anti-arrhythmias; anti-asthmatics; anti-cholesterolemics; anti-convulsants; anti-depressants; anti-diarrhea preparations; anti-histamines; antihypertensive drugs; anti-infectives; anti-inflammatory agents; anti-lipid agents; anti-manics; anti-nauseants; antistroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mucolytics; narcotics; neuroleptics; neuromuscular drugs; NSAIDS; peripheral vasodilators; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; and wound healing agents.

In some embodiments, Z is a therapeutic moiety obtained from a therapeutic agent selected from the group consisting of an antibiotic, an anti-inflammatory agent, an anti-viral agent, an anti-cancer agent, an anti-infective agent, and combinations thereof. In some embodiments, Z is a therapeutic moiety obtained from a corticosteroid. Non-limiting examples of corticosteroids include dexamethasone, prednisone, prednisolone, cortisone, hydrocortisone, betamethasone, and combinations thereof. In certain embodiments, Z is a dexamethasone residue. In some embodiments, Z is a therapeutic moiety obtained from an antibiotic. In certain embodiments, Z is a mithramycin residue.

In some embodiments, a compound having a formula (VIII) to (X) or a pharmaceutically acceptable salt thereof is presented.

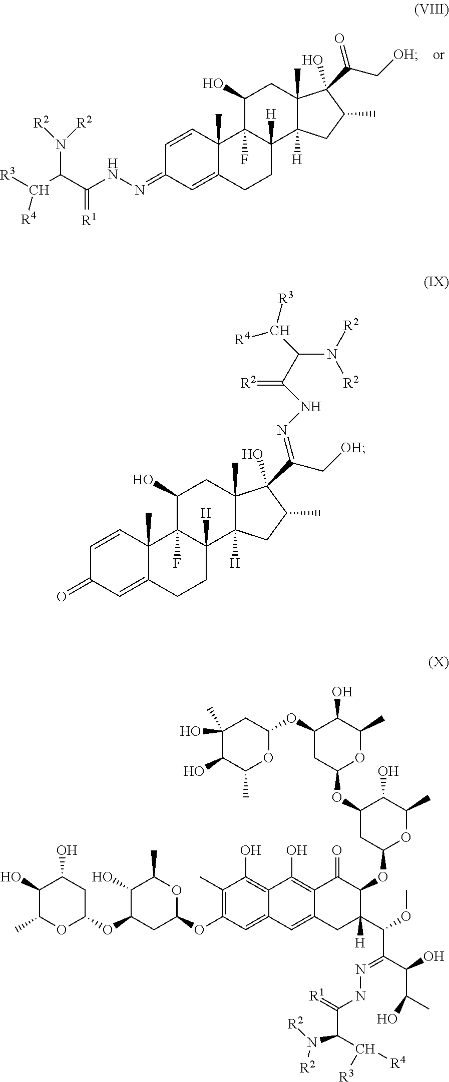

wherein R¹ is O or S;
R² is independently at each occurrence hydrogen or a C₁-C₃ alkyl group
R³ is hydrogen or a C₁-C₃ alkyl group; and
R⁴ is a C₁-C₆ alkyl group, a C₆-C₁₀ aryl group, or a C₅-C₁₀ heteroaryl group.

In accordance with embodiments of the present invention, the prodrug compounds of formula (I) to (X) are capable of releasing the therapeutic moiety Z after specifically binding to a target wherein the Z═N bond functions as a cleavable moiety. Further, the prodrug compounds of formula (I) to (X) of the present invention are capable of effectively transporting the therapeutic moiety across a blood brain barrier.

In some embodiments, a pharmaceutical composition is presented. The pharmaceutical composition includes a compound as described herein above or a pharmaceutically acceptable salt, solvate, hydrate, polymorph, or co-crystal thereof, and a pharmaceutical carrier, diluent, or excipient. In some embodiments, the pharmaceutical composition includes a compound having a formula (I).

The pharmaceutical compositions of the present invention may be in any form that allows for the composition to be administered to a subject. For example, the composition may be in the form of a solid, liquid, or gas (aerosol). Pharmaceutical compositions may be formulated to allow the active ingredients contained therein to be bioavailable upon administration of the composition to a subject. To further optimize the pharmacokinetic profile of the compounds of the present invention, the compounds may be administered in conjunction with a suitable delivery vehicle (e.g., microcapsules, microspheres, biodegradable polymer films, lipid-based delivery systems such as liposomes and lipid foams, viscous instillations and absorbable mechanical barriers) useful for maintaining the necessary concentrations of the prodrugs or the therapeutic agent at the site of the disease.

A process for preparing a pharmaceutical composition is also presented. The process includes mixing a compound as described hereinabove or a pharmaceutically acceptable salt, solvate, hydrate, polymorph, or co-crystal thereof with a pharmaceutically acceptable carrier, diluent, or excipient. In some embodiments, the process includes mixing a compound having a formula (I) or a pharmaceutically acceptable salt, solvate, hydrate, polymorph, or co-crystal thereof with a pharmaceutically acceptable carrier, diluent, or excipient.

In some embodiments, methods of treating or reducing symptoms of a certain disease by administering a compound of the present invention are also presented. The compounds or derivatives thereof can be administered to any host, including a human, a non-human animal, and mammals, in an amount effective to treat a disorder.

In some embodiments, a method of treating a brain disease is presented. The method includes administering to a patient an effective amount of a pharmaceutical composition including a compound of the present invention or a pharmaceutically acceptable salt, solvate, hydrate, polymorph, or co-crystal thereof. In some embodiments, the method includes administering to a patient an effective amount of a pharmaceutical composition including a compound of formula (I) or a pharmaceutically acceptable salt, solvate, hydrate, polymorph, or co-crystal thereof. In some embodiments, the brain disease is glioblastoma, glioma, medulloblastoma, or metastatic brain disease. In accordance with embodiments of the present invention, the compound of formula (I) is capable of effectively transporting the therapeutic moiety Z across a blood brain barrier.

The pharmaceutical composition may be administered by any suitable method known to a person skilled in the art. Typical routes of administration include, without limitation, oral, topical, parenteral, sublingual, rectal, vaginal, ocular, and intranasal. The term parenteral as used herein includes intravenous, intraperitoneal, intramuscular, intradermal, and epidermal including subcutaneous and intradermal, oral, or application to mucosal surfaces, e.g, by intranasal administration using inhalation of aerosol suspensions, and by implanting to muscle or other tissue in the subject.

### SPECIFIC EMBODIMENTS

The following enumerated embodiments are representative of some aspects of the invention.

Embodiment 1. A compound having a formula (I) or a pharmaceutically acceptable salt thereof:

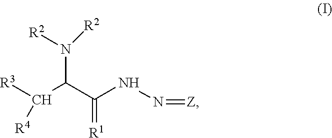

wherein Z is a therapeutic moiety;
R¹ is O or S;
R² is independently at each occurrence hydrogen or a C₁-C₃ alkyl group;
R³ is hydrogen or a C₁-C₃ alkyl group; and
R⁴ is a C₁-C₆ alkyl group, a C₆-C₁₀ aryl group, or a C₅-C₁₀ heteroaryl group.

Embodiment 2. The compound of embodiment 1, wherein the compound is an L and syn isomer of formula (I).

Embodiment 3. The compound of embodiment 1, wherein the compound is an L and anti isomer of formula (I).

Embodiment 4. The compound of embodiment 1, wherein the compound is a D and syn isomer of formula (I).

Embodiment 5. The compound of embodiment 1, wherein the compound is a D and anti isomer of formula (I).

Embodiment 6. The compound of anyone of embodiments 1-3, wherein the compound has a formula (II) or a pharmaceutically acceptable salt thereof:

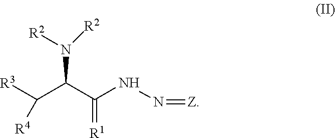

Embodiment 7. The compound of any one of embodiments 1-6, wherein the compound has a formula (III) to (VI) or a pharmaceutically acceptable salt thereof:

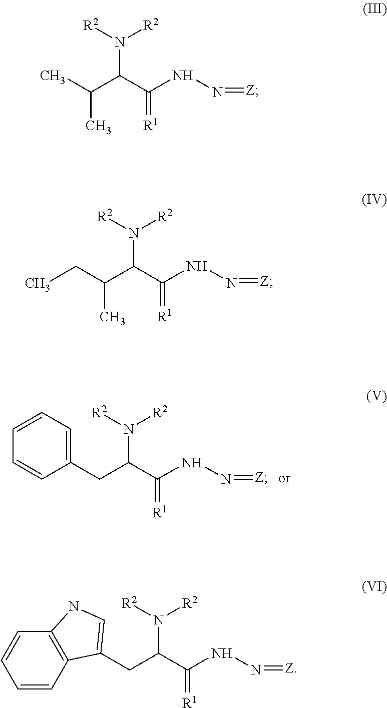

Embodiment 8. The compound of any one of embodiments 1-7, wherein Z is a therapeutic moiety obtained from a therapeutic agent selected from the group consisting of an antibiotic, an anti-inflammatory agent, an anti-viral agent, an anti-cancer agent, an anti-infective agent, and combinations thereof.

Embodiment 9. The compound of any one of embodiments 1-8, wherein Z is a therapeutic moiety obtained from a corticosteroid selected from the group consisting of dexamethasone, prednisone, prednisolone, triamcinolone, cortisone, hydrocortisone, and betamethasone.

Embodiment 10. The compound of any one of embodiments 1-9, wherein Z is a dexamethasone residue.

Embodiment 11. The compound of any one of embodiments 1-9, wherein Z is mithramycin residue.

Embodiment 12. A pharmaceutical composition comprising a compound of any one of embodiments 1-11 or a pharmaceutically acceptable salt, solvate, hydrate, polymorph, or co-crystal thereof, and a pharmaceutically carrier, diluent, or excipient.

Embodiment 13. A method of treating a brain disease comprising administering to a patient an effective amount of a pharmaceutical composition comprising a compound of formula (I) or a pharmaceutically acceptable salt thereof:

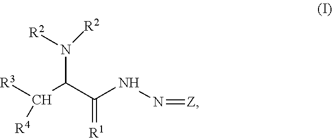

wherein Z is a therapeutic moiety;
R¹ is O or S;
R² is independently at each occurrence hydrogen or a C₁-C₃ alkyl group;
R³ is hydrogen or a C₁-C₃ alkyl group; and
R⁴ is a C₁-C₆ alkyl group, a C₆-C₁₀ aryl group, or a C₅-C₁₀ heteroaryl group.

Embodiment 14. The method of embodiment 14 wherein the compound is an L and syn isomer of formula (I).

Embodiment 15. The method of embodiment 14 wherein the compound is an L and anti isomer of formula (I).

Embodiment 16. The method of embodiment 14, wherein the compound is a D and syn isomer of formula (I).

Embodiment 17. The method of embodiment 14, wherein the compound is a D and anti isomer of formula (I).

Embodiment 18. The method of any one of embodiments 13-15, wherein the compound has a formula (II) or a pharmaceutically acceptable salt thereof:

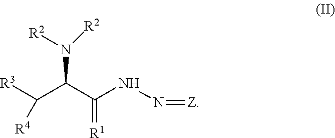

Embodiment 19. The method of any one of embodiments 1-18, wherein the compound has a formula (III) to (VI) or a pharmaceutically acceptable salt thereof:

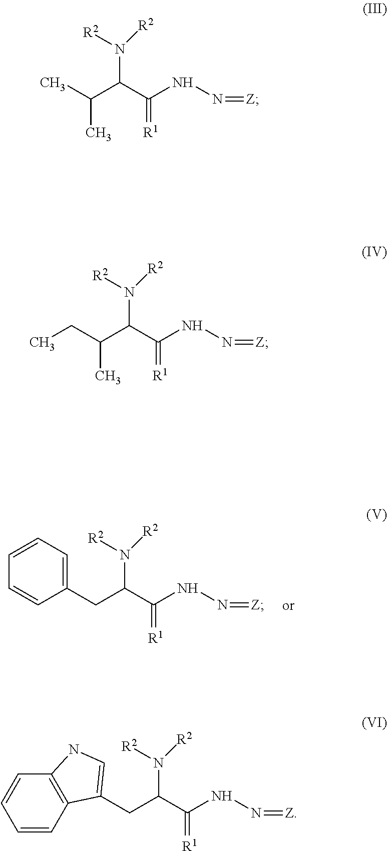

Embodiment 20. The method of any one of embodiments 1-19, wherein the brain disease comprises glioblastoma, medulloblastoma, glioma, or a brain metastatic disease.

### EXAMPLES

### General Methods for the Synthesis of Prodrugs

Dexamethasone (>95% purity) and Mithramycin A (>95% purity) were purchased from a commercial source and used for the conjugation with linkers. All commercial reagents and solvents were used as received. Reaction progress, intermediates and final products were monitored and assessed using LC/MS, HPLC-UV and by NMR (1H and 13C). Purification of the prodrug was performed on a silica gel column using flash column chromatography or by prep-LC when required. The ¹H NMR spectra was recorded on a Bruker BioSpin GmbH spectrometer at 300 MHz. Coupling constants (J) are reported in hertz (Hz). Chemical shifts (δ) are reported in parts per million referenced with respect to the residual solvent (DMSO-d6) 2.50 ppm or from internal standard tetramethylsilane 0.00 ppm. The following abbreviations were used in the reporting spectra: s, singlet; d, doublet; t, triplet; q, quartet; m, multiplet; dd, doublet of doublets; td, triplet of doublets; ddd, doublet of doublet of doublets. All prodrugs synthesized in this project were >95% pure.

### Example 1 Synthesis of Compound I

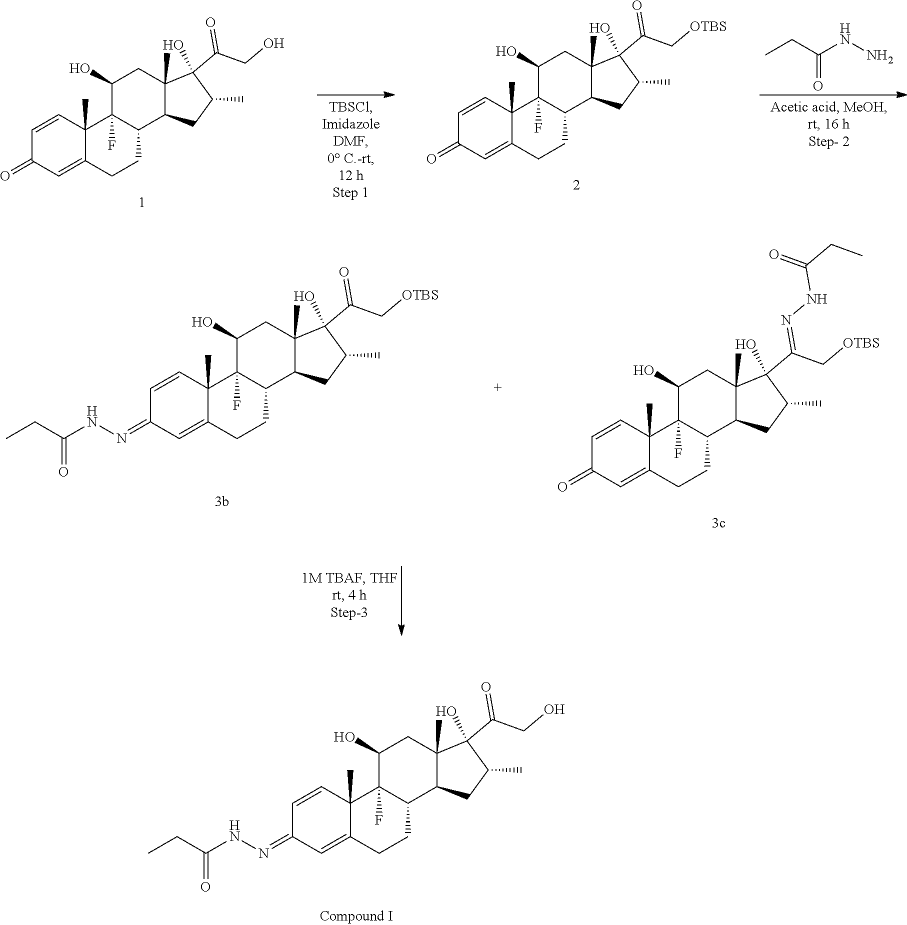

To a stirred solution of dexamethasone (5 g, 12.73 mmol) in DMF (25 mL) were added imidazole (1.73 g, 25.47 mmol) and TBDMSCl (2.30 g, 0.30 mmol) at 0° C. and the resulting mixture was stirred for 12 h at room temperature. The progress of the reaction was monitored by TLC. After completion of the reaction, ice water (50 mL) was added to the reaction mixture which resulted in the formation of a precipitate. The precipitate was filtered, washed with water and dried over vacuum to afford Compound-2 (5.2 g, 80%) as a white solid.

To a stirred solution of Compound-2 (3.0 g, 5.92 mmol) in methanol (12 mL) were added propionohydrazide (1.5 g, 17.78 mmol) and AcOH (177 mg, 2.96 mmol) at 0° C. The reaction mixture was stirred for 12 h at ambient temperature. After completion of the reaction, methanol was concentrated under reduced pressure and diluted with H₂O (200 mL), extracted with ethyl acetate (2×200 mL). The organic layer washed with brine (150 mL), dried over sodium sulphate and concentrated under reduced pressure to get crude compound. The crude compound was purified by flash column chromatography to get Compound-3b (800 mg, 23%) as a pale yellow solid.

To a stirred solution of Compound-3b (0.5 g, 0.86 mmol) in THF (5 mL) was added TBAF (1 M, 1.7 mL, 1.72 mmol) at 0° C. The reaction mixture stirred for 2 h at ambient temperature. After completion of the reaction, ice water (30 mL) added to the reaction mixture and extracted with ethyl acetate (2×150 mL). Organic layer was washed with brine (120 mL), dried over sodium sulphate and concentrated under reduced pressure to get crude compound 300 mg. Out of 300 mg crude compound, 100 mg was purified by preparative HPLC method and lyophilized. The compound was basified with saturated NaHCO₃ solution and extracted with ethyl acetate (50 mL), then concentrated under reduced pressure and lyophilized to afford Compound I (15 mg, yield: 11.27%) as a white solid.

1H NMR (400 MHz, DMSO-d6): δ 10.48 (dd, 1H), 6.74 (dd, 1H), 6.41 (dd, J=17.0, 10.1 Hz, 1H), 6.22 (dd, J=22.5, 10.0 Hz, 1H), 5.13 (dd, J=10.4, 2.8 Hz, 1H), 4.94 (s, 1H), 4.68 (t, J=5.8 Hz, 1H), 4.49 (dd, J=19.3, 5.9 Hz, 1H), 4.07 (dd, J=19.3, 5.8 Hz, 2H), 2.94 (m, 1H), 2.67-2.55 (m, 2H), 2.34-2.09 (m, 5H), 1.71 (m, 1H), 1.58 (m, 1H), 1.41 (d, J=5.2 Hz, 3H), 1.29 (m, 2H), 1.02 (q, J=7.4 Hz, 4H), 0.84 (s, 3H), 0.78 (d, J=7.2 Hz, 3H) ppm. LC-MS (ESI): m/z calculated 462, found 463 (M+H)+, (RT=5.1, 99.78% purity). HPLC: 99.31% (250 nm), 99.49% (295 nm).

### Example 2 Synthesis of Compound II

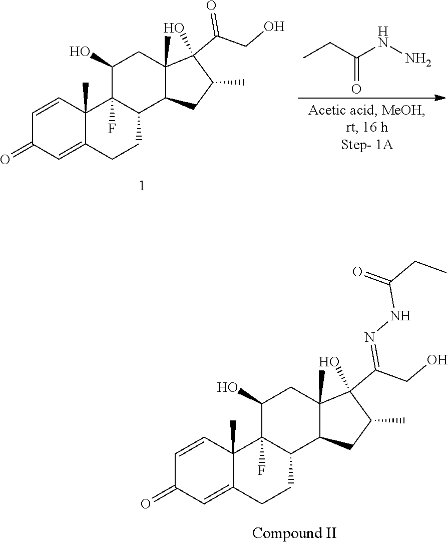

To a stirred solution of dexamethasone (1.0 g, 2.54 mmol) in methanol and DMSO (10 mL) were added propionohydrazide (0.67 g, 7.64 mmol) and AcOH (75 mg, 1.27 mmol) at 0° C. The reaction mixture was stirred for 12 h at 50° C. After completion of the reaction, methanol was evaporated under reduced pressure and diluted with ice cold water (50 mL) and extracted with ethyl acetate (2×100 mL). The organic layer washed with brine solution (50 mL) and the organic layer was dried over sodium sulphate, then concentrated under reduced pressure to get crude compound. The crude compound was purified by preparative HPLC method and lyophilized. The compound was basified with saturated NaHCO₃ solution and extracted with ethyl acetate (50 mL), then concentrated under reduced pressure and lyophilized to afford Compound II (26 mg, yield: 2.22%) as a white solid. ¹H NMR (400 MHz, DMSO-d₆): δ 10.54 (bd, 1H), 6.99 (bd, 10.1 Hz, 1H), 6.60 (bd, J=30.4, 10.3 Hz, 1H), 5.96 (d, J=24.5 Hz, 1H), 5.04 (d, J=86.4 Hz, 1H), 4.48 (d, J=19.2 Hz, 1H), 4.09 (s, 2H), 2.92 (m, 3H), 2.36-2.20 (m, 2H), 2.17-2.04 (m, 2H), 1.71-1.53 (m, 3H), 1.41 (d, J=6.3 Hz, 4H), 1.40-1.35 (m, 1H), 1.33-1.22 (m, 2H), 1.02 (dd, J=14.9, 7.5 Hz, 4H), 0.83 (s, 3H), 0.77 (d, J=7.2 Hz, 3H) ppm. LC-MS (ESI): m/z calculated 462, found 463 (M+H)f, (RT=5.3, 99.38% purity). HPLC: 98.15% (250 nm), 98.89% (290 nm).

### Example 3 Synthesis of Compounds IIIA and IIIB

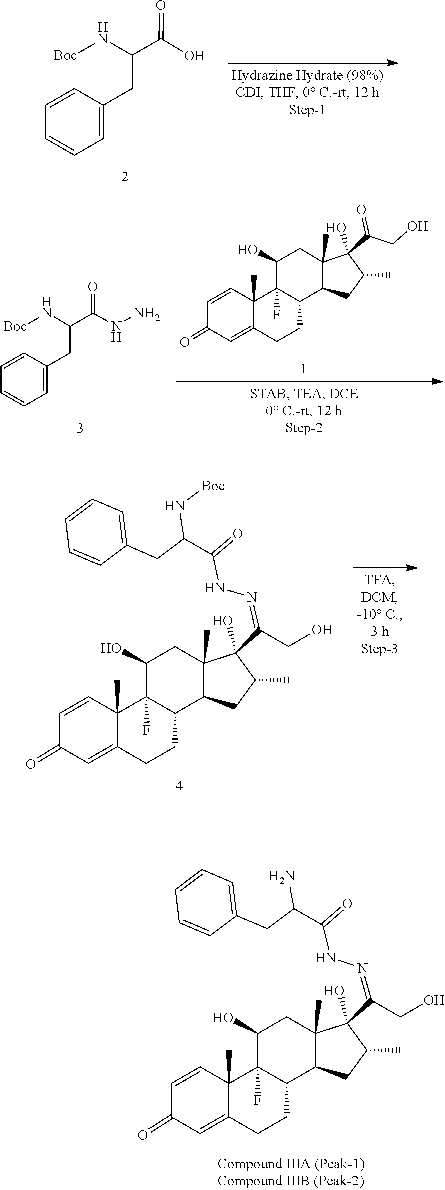

To a stirred solution of (tert-butoxycarbonyl)phenylalanine (2 g, 7.53 mmol) in THF (12 mL) was added CDI (1.35 g, 8.27 mmol) at rt and stirred for 1 h. After hydrazine hydride was added to the reaction mixture at 0° C. The resulting mixture was stirred for 12 h at room temperature and monitored by TLC. The reaction mixture was quenched with ice water (150 mL) and compound extracted with ethyl acetate (2×200 mL). The combined organic layer washed with brine solution and the organic layer was dried over sodium sulphate, then concentrated under reduced pressure to get crude. The crude material was purified by flash column chromatography and compound eluted with 5 to 6% of methanol in DCM gradient to afford Compound-3 (1.5 g, 71%) as an off white solid.

To a stirred solution of dexamethasone (1) (1 g, 2.55 mmol) and Compound-3 (1.5 g, 5.10 mmol) in DCE (10 mL) was slowly added TEA (0.55 ml, 3.82 mmol) and STAB (650 mg, 3.06 mmol) at 0° C. The resulting mixture was stirred for 12 h at ambient temperature and monitored by LC-MS. The reaction mixture was quenched with water (100 mL) and compound extracted DCM (2×150 mL). The combined organic layer was washed with brine solution and the organic layer was dried over sodium sulphate, then concentrated under reduced pressure to get crude. The crude material was purified by flash chromatography and compound eluted with 2 to 4% of methanol in DCM gradient to afford Compound-4 (1.2 g, 72%) as a white solid.

To a stirred solution of Compound-4 (400 mg, 0.61 mmol) in DCM (6 mL) was added slowly TFA (2 mL) at −10° C. The resulting mixture was stirred for 3 h at −10 to −5° C. and monitored by TLC & LC-MS. After completion of the reaction concentrated under reduced pressure to get the residue and triturated with diethyl ether to get the crude, the crude was purified by prep-HPLC method to afford the Compound IIIA (P1-30.8 mg) and Compound IIIB (P2-40 mg) as a white solid.

Compound IIIA (Peak1): 1H NMR (400 MHz, DMSO-d6): δ 10.48 (s, 1H), 7.37-7.11 (m, 6H), 6.22 (d, J=10.1 Hz, 1H), 6.00 (s, 1H), 5.54 (d, J=87.6 Hz, 1H), 5.29 (dd, J=12.6, 4.2 Hz, 1H), 4.53 (d, J=56.8 Hz, 1H), 4.35-4.05 (m, 3H), 3.51 (dd, J=9.4, 4.0 Hz, 1H), 3.18 (s, 1H), 3.08 (dd, J=13.5, 4.2 Hz, 1H), 2.95 (dd, J=13.4, 4.3 Hz, 1H), 2.72-2.57 (m, 2H), 2.39-2.25 (m, 3H), 2.21-2.05 (m, 2H), 1.78 (s, 1H), 1.65-1.51 (m, 1H), 1.50-1.38 (m, 4H), 1.38-1.19 (m, 2H), 1.14-1.02 (m, 1H), 0.96-0.77 (m, 6H). LC-MS (ESI): m/z calculated 553, found 554 (M+H)+, (RT=4.45 & 99.69% purity). HPLC: 99.08% (230 nm), 98.84% (240 nm), M.R: 184 0C-188 0C.

Compound IIIB (Peak2): 1H NMR (400 MHz, DMSO-d6): δ 10.49 (s, 1H), 7.37-7.15 (m, 6H), 6.23 (t, J=9.7 Hz, 1H), 6.00 (s, 1H), 5.69-5.36 (m, 1H), 5.28 (s, 1H), 4.53 (d, J=65.7 Hz, 1H), 4.29 (d, J=3.4 Hz, 1H), 4.25-4.15 (m, 1H), 4.10 (dd, J=23.5, 17.2 Hz, 2H), 3.56-3.42 (m, 1H), 3.17 (s, 1H), 3.13-3.05 (m, 1H), 2.97 (dd, J=13.2, 4.6 Hz, 1H), 2.72-2.54 (m, 4H), 2.33 (s, 4H), 2.19-2.05 (m, 3H), 1.77 (s, 1H), 1.71-1.53 (m, 1H), 1.45 (dd, J=22.6, 10.9 Hz, 5H), 1.34 (d, J=9.2 Hz, 1H), 1.23 (s, 1H), 1.07 (s, 2H), 0.96-0.84 (m, 5H), 0.81 (d, J=7.1 Hz, 1H). LC-MS (ESI): m/z calculated 553, found 554 (M+H)+, (RT=4.5 & 99.58% purity). HPLC: 99.07% (230 nm), 98.99% (240 nm). M.R: 184 0C-188 0C.

### Example 4: Synthesis of Compounds IVA and IVB

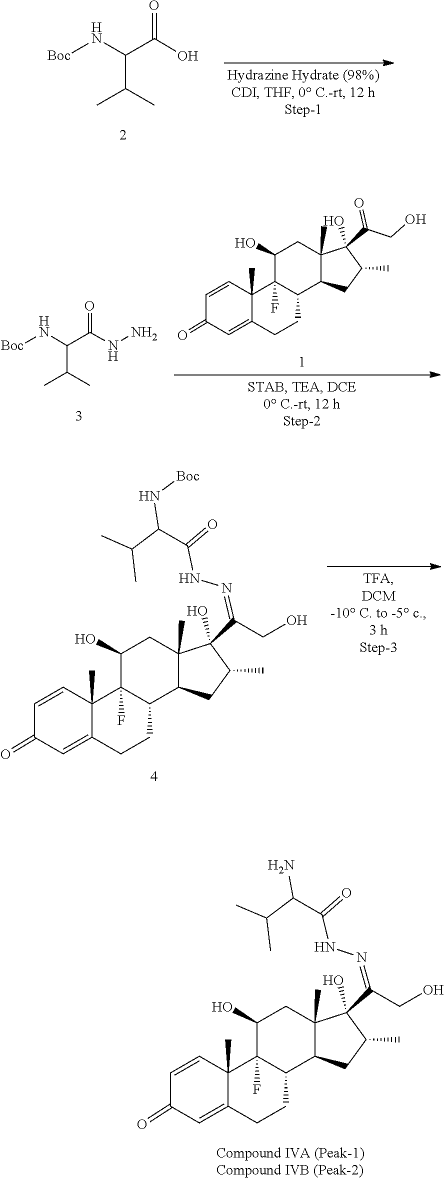

To a stirred solution of (tert-butoxycarbonyl)valine (2 g, 9.20 mmol) in THF (12 mL) was added CDI (1.64 g, 10.12 mmol) at rt and stirred for 1 h. After hydrazine hydride (0.88 g, 27.6 mmol) was added to the reaction mixture at 0° C. The resulting mixture was stirred for 12 h at room temperature and monitored by TLC. The reaction mixture was quenched with ice water (100 mL) and compound extracted with ethyl acetate (2×150 mL). The combined organic layer washed with brine solution and the organic layer was dried over sodium sulphate, then concentrated under reduced pressure to get the crude. The crude material was purified by flash column chromatography and compound eluted with 4 to 6% of methanol in DCM gradient to afford Compound-3 (1.2 g, 56%) as an off white solid.

To a stirred solution of dexamethasone (1) (1 g, 2.54 mmol) and Compound-3 (590 mg, 2.54 mmol) in DCE (10 mL) were added slowly TEA (0.35 ml, 2.54 mmol) and STAB (538 mg, 2.54 mmol) at 0° C. The resulting mixture was stirred for 12 h at ambient temperature and monitored by TLC. The reaction mixture was quenched with water (100 mL) and compound extracted DCM (2×150 mL). The combined organic layer was washed with brine solution and the organic layer was dried over sodium sulphate, then concentrated under reduced pressure to get crude. The crude material was purified by flash column chromatography and compound eluted with 2 to 4% of methanol in DCM gradient to afford Compound-4 (870 mg, 56%) as an off white solid.

To a stirred solution of Compound-4 (400 mg, 0.66 mmol) in DCM (10 mL) was added slowly TFA (2 mL) at 0° C. The resulting mixture was stirred for 3 h at ambient temperature and monitored by TLC. After completion of the reaction concentrated under reduced pressure to get the residue and triturated with diethyl ether to get the crude, the crude was purified by prep-HPLC method to afford the Compound IVA (P1-28 mg) & Compound IV (P2-26 mg) as a white solid.

Compound IVA (Peak 1): 1H NMR (400 MHz, DMSO-d6): δ 10.84 (s, 1H), 7.33 (d, J=10.1 Hz, 1H), 6.23 (d, J=10.1 Hz, 1H), 6.02 (s, 1H), 5.75 (d, J=40.7 Hz, 1H), 5.34 (dd, J=27.7, 4.1 Hz, 1H), 4.64 (d, J=30.9 Hz, 1H), 4.31 (dd, J=33.6, 14.9 Hz, 2H), 4.12 (s, 1H), 3.09 (s, 1H), 2.62 (d, J=5.1 Hz, 1H), 2.30 (dd, J=24.2, 6.0 Hz, 2H), 2.09 (ddd, J=25.1, 15.2, 6.6 Hz, 3H), 1.77 (s, 1H), 1.64-1.45 (m, 5H), 1.35 (dd, J=13.1, 4.5 Hz, 1H), 1.08 (d, J=8.4 Hz, 1H), 1.05-0.77 (m, 12H). LC-MS (ESI): m/z calculated 505, found 506 (M+H)+, (RT=4.18 & 99.85% purity). HPLC: 99.14% (230 nm), 99.52% (240 nm), M.R: 226 0C-230 0C.

Compound IVB (Peak 2): 1H NMR (400 MHz, DMSO-d6): δ10.88 (s, 1H), 7.33 (d, J=10.1 Hz, 1H), 6.23 (dd, J=10.1, 1.5 Hz, 1H), 6.01 (s, 1H), 5.80 (t, J=41.7 Hz, 1H), 5.34 (dd, J=9.5, 4.3 Hz, 1H), 4.79-4.56 (m, 1H), 4.39-4.05 (m, 4H), 3.23-3.96 (m, 1H), 2.60 (d, J=13.6 Hz, 1H), 2.40-2.25 (m, 2H), 2.23-2.00 (m, 3H), 1.78 (dd, J=10.7, 5.8 Hz, 1H), 1.66-1.44 (m, 5H), 1.41-1.29 (m, 1H), 1.17-1.09 (m, 1H), 1.07-0.77 (m, 12H). LC-MS (ESI): m/z calculated 505, found 506 (M+H)+, (RT=4.16 & 99.82% purity). HPLC: 99.69% (230 nm), 99.41% (240 nm). M.R: 226 0C-230 0C

### Example 5: Synthesis of Compounds VA and VB

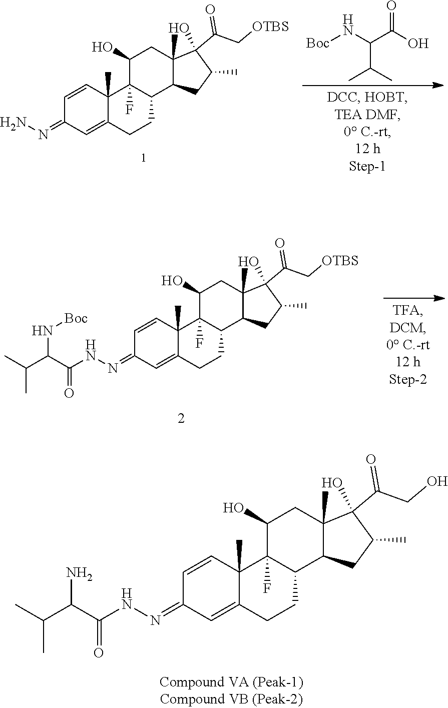

To a stirred solution of (tert-butoxycarbonyl)valine (209 mg, 0.96 mmol) and Compound-1 (500 mg, 0.96 mmol) in DMF (5 mL) were added TEA (0.34 mL, 2.43 mmol), HOBT (208 mg, 1.53 mmol) and DCC (317 mg, 1.53 mmol) at 0° C. The resulting mixture was stirred for 12 h at room temperature and monitored by TLC. After completion of the reaction quenched with cold water (100 mL) and extracted with DCM (2×150 mL). The combined organic layer washed with brine solution and the organic layer was dried over sodium sulphate, then concentrated under reduced pressure to get crude. The crude material was purified by flash column chromatography and compound eluted with 3 to 4% methanol in DCM gradient to afford the Compound-2 (620 mg, 89%) as an off white solid.

To a stirred solution of Compound-2 (400 mg, 0.55 mmol) in DCM (6 mL) was slowly added TFA (2 ml) at 0° C. and the resulting mixture was stirred for 12 h at room temperature. The reaction was monitored by TLC and after completion of the reaction. The reaction mixture was concentrated under reduced pressure to get residue, basified with sat NaHCO₃ solution, and extracted with ethyl acetate (2×100 mL). The combined organic layer was dried over sodium sulphate, then concentrated under reduced pressure to get the crude product. The crude product was purified by prep-HPLC to afford the Compound VA (P1-14 mg) & Compound VB (P2-15 mg) as an off white solid.

Compound VA (Peak1):1H NMR (400 MHz, DMSO-d6): δ 11.13 (s, 1H), 6.73 (d, J=62.1 Hz, 1H), 6.50 (dd, J=17.5, 10.2 Hz, 1H), 6.27 (dd, J=30.7, 11.3 Hz, 1H), 5.21-5.10 (m, 1H), 4.95 (d, J=2.4 Hz, 1H), 4.69 (t, J=5.8 Hz, 1H), 4.49 (dd, J=19.3, 5.9 Hz, 1H), 4.32 (d, J=4.8 Hz, 1H), 4.07 (dd, J=19.2, 5.6 Hz, 2H), 3.56 (d, J=6.1 Hz, 1H), 2.94 (s, 1H), 2.61 (d, J=8.9 Hz, 1H), 2.24 (d, J=8.9 Hz, 1H), 2.13 (d, J=8.7 Hz, 3H), 2.04-1.96 (m, 1H), 1.74 (d, J=5.8 Hz, 1H), 1.67-1.53 (m, 1H), 1.42 (d, J=7.2 Hz, 4H), 1.34 (d, J=11.8 Hz, 1H), 1.06 (s, 1H), 0.99-0.86 (m, 6H), 0.85 (s, 3H), 0.78 (d, J=7.2 Hz, 3H). LC-MS (ESI): m/z calculated 505, found 506 (M+H)+, (RT=4.14, 4.28 & 95.19, 4.51% purity). HPLC: 95.64 & 3.34% (250 nm), 96.88 & 2.80% (300 nm), M.R: >240 0C.

Compound VB (Peak 2): 1H NMR (400 MHz, DMSO-d6): δ 11.13 (s, 1H), 6.84-6.61 (m, 1H), 6.49 (t, J=11.8 Hz, 1H), 6.26 (dd, J=29.3, 10.2 Hz, 1H), 5.16 (d, J=9.5 Hz, 1H), 4.95 (d, J=3.2 Hz, 1H), 4.70 (s, 1H), 4.50 (dd, J=19.1, 5.8 Hz, 1H), 4.28-4.00 (m, 2H), 3.47-3.39 (m, 1H), 2.94 (s, 1H), 2.61 (s, 1H), 2.24 (d, J=9.4 Hz, 1H), 2.13 (d, J=13.9 Hz, 2H), 2.00-1.88 (m, 1H), 1.74 (s, 1H), 1.61 (d, J=11.0 Hz, 1H), 1.43 (d, J=7.5 Hz, 4H), 1.35 (d, J=10.0 Hz, 1H), 1.06 (s, 1H), 0.96 (d, J=6.9 Hz, 1H), 0.93-0.83 (m, 7H), 0.78 (d, J=7.2 Hz, 3H). LC-MS (ESI): m/z calculated 505, found 506 (M+H)+, (RT=4.14, 4.26 & 92.5, 6.9% purity). HPLC: 91.71 & 5.08% (250 nm), 92.21 & 5.23% (300 nm), M.R: >240 0C.

### Example 6: Synthesis of Compounds VIA and VIB

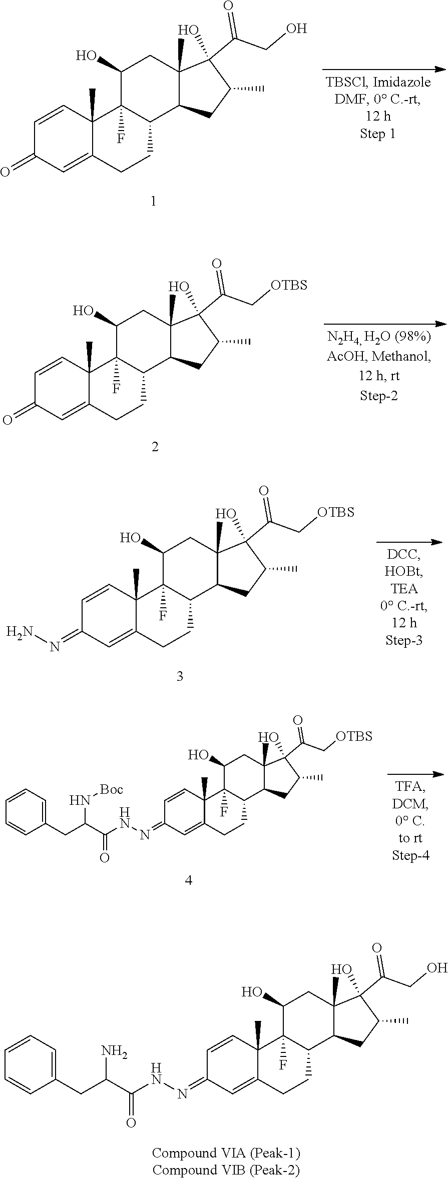

To a stirred solution of dexamethasone (5 g, 12.73 mmol) in DMF (25 mL) were added imidazole (1.73 g, 25.47 mmol) and TBDMSCl (2.30 g, 0.30 mmol) at 0° C. and the resulting mixture was stirred for 12 h at room temperature. The progress of the reaction was monitored by TLC. After completion of the reaction, ice cold water (50 mL) was added to the reaction mixture which resulted in the formation of a precipitate. The precipitate was filtered, washed with water and dried over vacuum to afford Compound-2 (5.2 g, 80%) as a white solid.

To a stirred solution of Compound-2 (5 g, 9.88 mmol) in MeOH (50 mL) were added acetic acid (0.28 mL, 2.667 mmol) and hydrazine hydrate (1.48 g, 29.64 mmol) at 0° C. and the resulting mixture was stirred for 3 h at room temperature. After completion of the reaction, the solvent was removed and the resulting residue was diluted with water (250 mL) and extracted with ethyl acetate (2×400 mL). The combined organic layer was washed with brine solution (300 mL) and dried over sodium sulphate, then concentrated under reduced pressure to get the crude product. The crude was purified by flash column chromatography and pure compound eluted in 70% of ethyl acetate in pet ether gradient to

To a stirred solution of Compound-3 (500 mg, 0.96 mmol) and (tert-butoxycarbonyl)phenylalanine (255 mg, 0.96 mmol) in DMF (5 mL) and were added slowly TEA (0.34 mL, 2.43 mmol), HOBT (208 mg, 1.53 mmol) and DCC (317 mg, 1.53 mmol) at 0° C. The resulting mixture was stirred at ambient temperature for 12 h and monitored by TLC. After completion of the reaction, quenched with cold water (50 mL) and compound extracted with ethyl acetate (2×100 mL). The combined organic layer washed with brine solution and the organic layer was dried over sodium sulphate, then concentrated under reduced pressure to get crude. The crude material was purified by flash chromatography and compound eluted with 30 to 35% of ethyl acetate in pet ether gradient to afford the Compound-3 (480 mg, 65%) as a pale yellow solid.

To a stirred solution of Compound-3 (300 mg, 0.39 mmol) in DCM (6 mL) at 0° C. was added TFA (2 mL) and the reaction mixture was stirred for 12 h at ambient temperature. The reaction was monitored by TLC. After completion of the reaction and reaction mixture was concentrated to get the residue. Water was added and basified with sat NaHCO₃ solution and extracted with ethyl acetate. The combined organic layer was washed with brine solution. The organic layer was dried over sodium sulphate, then concentrated under reduced pressure to get crude. The crude was purified by prep HPLC to afford Compound VIA (P1-17 mg) & Compound VIB (P2-19 mg) as white solids.

Compound VIA (Peak 1): 1H NMR (400 MHz, DMSO-d6): δ 11.19 (s, 1H), 7.34-7.22 (m, 6H), 6.77 (s, 1H), 6.52 (t, J=10.8 Hz, 1H), 6.32-6.24 (m, 1H), 5.17 (s, 1H), 4.96 (d, J=5.2 Hz, 1H), 4.71-4.66 (m, 1H), 4.53-4.46 (m, 1H), 4.11-4.05 (m, 2H), 3.14-2.93 (m, 4H), 2.63-2.62 (m, 1H), 2.26-2.11 (m, 4H), 1.76-1.74 (m, 1H), 1.66-1.57 (m, 1H), 1.43-1.42 (m, 4H), 1.36-1.29 (m, 1H), 1.08-1.06 (m, 1H), 0.85 (s, 3H), 0.79 (d, J=6.8 Hz, 3H). LC-MS (ESI): m/z calculated 553, found 554 (M+H)+, (RT=4.35 & 96.98% purity). HPLC: 96.86% (250 nm), 97.35% (300 nm). M.R: >240 0C.

Compound VIB (Peak 2): 1H NMR (400 MHz, DMSO-d6): δ 11.17 (s, 1H), 7.35-7.24 (m, 6H), 6.81 (s, 1H), 6.54-6.27 (m, 2H), 5.17 (s, 1H), 4.95 (s, 1H), 4.71-4.68 (m, 1H), 4.61 (dd, J1=7.2 Hz, J2=3.6 Hz, 1H), 4.10-4.04 (m, 3H), 3.05-2.88 (m, 3H), 2.67-2.60 (m, 1H), 2.33-2.10 (m, 4H), 1.76-1.73 (m, 1H), 1.65-1.56 (m, 1H), 1.43-1.17 (m, 5H), 1.07-1.05 (m, 1H), 0.85 (s, 3H), 0.78 (d, J=6.4 Hz, 3H). LC-MS (ESI): m/z calculated 553, found 554 (M+H)+, (RT=4.4 & 94.83% purity). HPLC: 92.31% (250 nm), 92.65% (300 nm). M.R: >240 0C.

### Synthesis of Compounds VIIA and VIIB

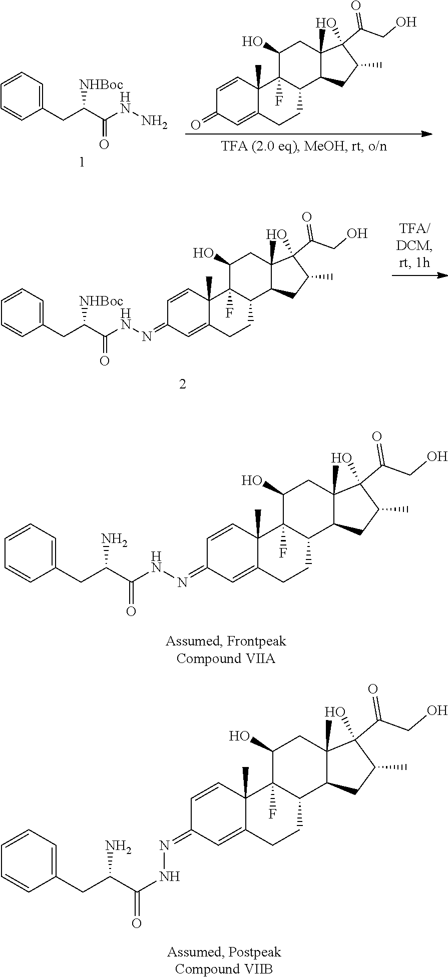

To a stirred solution of compound 1 (5.0 g, 17.9 mmol, 1 equiv) and dexamethasone (7.0 g, 17.9 mmol, 1 equiv) in MeOH (20 mL) was added TFA (2.6 mL, 35.8 mmol, 2 equiv) at room temperature. The resulting mixture was stirred overnight at room temperature. The resulting mixture was concentrated under reduced pressure. The residue was purified by silica gel column chromatography, eluted with PE/EA (1:1) to afford compound 2 (10 g, 76.91%) as a white solid. MS (ESI)(m/z): [M+1]+ calculated: 654.3; found: 654.4.

To a stirred solution of compound 2 (10 g, 15.3 mmol, 1 equiv) in DCM (75 mL) was added TFA (25 mL) at room temperature. The resulting mixture was stirred for 1 h at room temperature. The resulting mixture was concentrated under vacuum. The residue was purified by reversed-phase flash chromatography with the following conditions: column, C18 silica gel; mobile phase, MeCN in Water (10 mmol/L NH4HCO3), 20% to 60% gradient in 30 min; detector, UV 254 nm. The resulting mixture was concentrated under reduced pressure to afford product (mixture, ratio: ~1:1, 2.7 g) as white solid. The product (mixture, ratio: ~1:1, 2.7 g) was purified by Chiral-Prep-HPLC with the following conditions (Column: CHIRAL ART Cellulose-SB, 2*25 cm, 5 μm; Mobile Phase A: MtBE(0.5% 2 M NH3-MeOH)—HPLC, Mobile Phase B: EtOH—HPLC; Flow rate: 20 mL/min; Gradient: 30% B to 30% B in 7.5 min; Wave Length: 220/254 nm; Frontpeak (min): 4.63; Postpeak (min): 6.65; Sample Solvent: EtOH—HPLC; Injection Volume: 0.7 mL; Number Of Runs: 10) to afford Frontpeak: Compound VIIA (1.0877 g, 45.48%) as an off-white solid and Postpeak: Compound VIIB (1.0355 g, 43.25%) as an off-white solid.

Compound VIIA: 1H NMR (300 MHz, DMSO-d6) δ 10.73 (s, 1H), 7.32-7.14 (m, 5H), 6.50-6.40 (m, 2H), 6.26 (ddd, J=9.1, 7.1, 1.9 Hz, 1H), 5.20-5.12 (m, 1H), 4.98-4.93 (m, 1H), 4.70 (s, 1H), 4.50 (dd, J=19.1, 4.9 Hz, 1H), 4.11 (s, 2H), 3.61 (ddd, J=7.9, 6.0, 2.0 Hz, 1H), 2.94 (s, 2H), 2.98-2.87 (m, 1H), 2.68 (dd, J=13.2, 8.0 Hz, 1H), 2.17 (dp, J=31.4, 12.0, 11.3 Hz, 4H), 1.72 (s, 2H), 1.61 (q, J=11.9 Hz, 1H), 1.42 (s, 3H), 1.38 (d, J=14.7 Hz, 1H), 1.07 (ddd, J=11.8, 8.0, 4.1 Hz, 1H), 0.85 (s, 3H), 0.78 (d, J=7.2 Hz, 3H). MS (ESI)(m/z) [M+H]+ calculated: 554.3; found: 554.3.

Compound VIIB: 1H NMR (300 MHz, DMSO-d6) δ 10.66 (s, 1H), 7.23 (ddt, J=22.0, 14.6, 6.8 Hz, 5H), 6.50-6.40 (m, 2H), 6.26 (ddd, J=9.3, 7.1, 1.8 Hz, 1H), 5.19-5.12 (m, 1H), 4.95 (d, J=3.3 Hz, 1H), 4.70 (t, J=5.8 Hz, 1H), 4.50 (dd, J=19.2, 5.9 Hz, 1H), 4.16-4.02 (m, 2H), 3.64-3.56 (m, 1H), 2.93 (dd, J=13.4, 5.9 Hz, 2H), 2.63 (ddd, J=31.1, 13.9, 8.2 Hz, 1H), 2.23 (t, J=13.9 Hz, 1H), 2.17-2.05 (m, 4H), 1.61 (q, J=11.7 Hz, 1H), 1.42 (s, 3H), 1.34 (d, J=8.7 Hz, 3H), 1.12-1.01 (m, 1H), 0.85 (s, 3H), 0.78 (d, J=7.2 Hz, 3H). MS (ESI)(m/z) [M+H]+ calculated: 554.3; found: 554.3.

### Synthesis of Compounds VIIIA and VIIIB

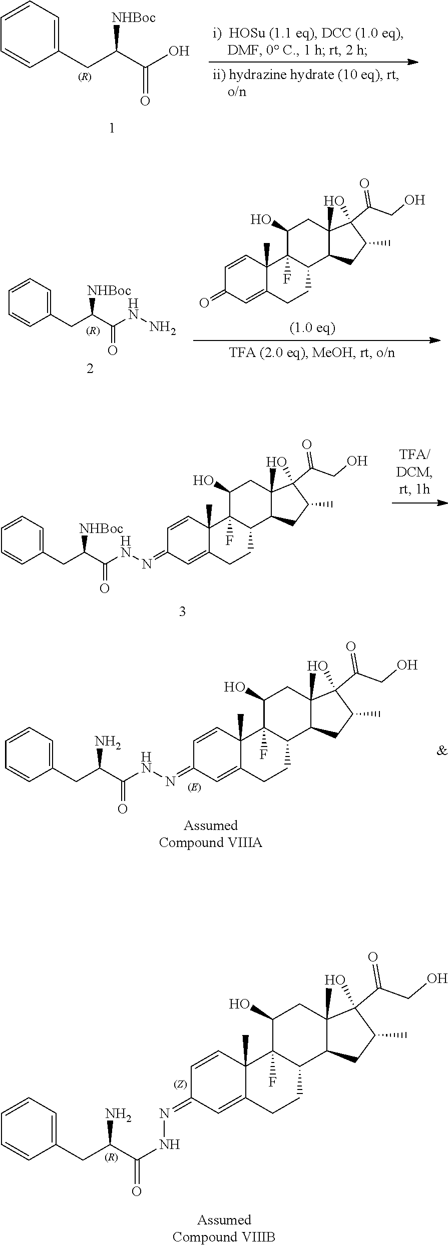

To a stirred solution of compound 1(2.0 g, 7.5 mmol, 1 equiv) and 1-hydroxypyrrolidine-2,5-dione (0.9 g, 8.3 mmol, 1.1 equiv) in DMF (20 mL) were added DCC (1.5 g, 7.5 mmol, 1.0 equiv) at 0° C. The resulting mixture was stirred for 1 h at 0° C. and 2 hours at room temperature. To the above mixture was added hydrazine hydrate (3.7 g, 75.3 mmol, 10.0 equiv) dropwise at room temperature. The resulting mixture was stirred overnight at room temperature. The resulting mixture was filtered, the filter cake was washed with EtOAc (2×50 mL). The filtrate was concentrated under reduced pressure. The resulting mixture was diluted with EtOAc (100 mL). The combined organic layers were washed with KHCO₃ (sat. 2×100 mL), dried over anhydrous Na₂SO₄. After filtration, the filtrate was concentrated under reduced pressure. This resulted compound 2 (2.1 g, 90.7%) as a white solid. MS (ESI)(m/z): [M+1]⁺ calculated: 280.2; found: 280.3.

To a stirred solution of compound 2 (2.1 g, 7.5 mmol, 1 equiv) and dexamethasone (2.9 g, 7.5 mmol, 1.00 equiv) in MeOH (20 mL) were added TFA (1.1 mL, 15.0 mmol, 2.0 equiv) at room temperature. The resulting mixture was stirred overnight at room temperature. The resulting mixture was concentrated under reduced pressure. The residue was purified by reversed-phase flash chromatography with the following conditions: column, C18 silica gel; mobile phase, MeCN in Water (0.1% FA), 20% to 70% gradient in 30 min; detector, UV 254 nm. The resulting mixture was concentrated under reduced pressure. This resulted in compound 3(3.1 g, 61.2%) as a light yellow solid. MS (ESI)(m/z): [M+1] calculated: 654.3; found: 654.4.

To a stirred solution of compound 3 (3.0 g, 4.6 mmol, 1 equiv) in DCM (30 mL) were added TFA (10 mL) at room temperature. The resulting mixture was stirred for 1 h at room temperature. The resulting mixture was concentrated under vacuum. The residue was purified by reversed-phase flash chromatography with the following conditions: column, C18 silica gel; mobile phase, MeCN in Water (10 mmol/L NH₄HCO₃), 30% to 50% gradient in 30 min; detector, UV 254 nm. The resulting mixture was concentrated under reduced pressure to afford product (mixture, ratio: ~3:2, 2.5 g) as white solid. The product (mixture, ratio: ~3:2, 2.5 g) was purified by Chiral-Prep-HPLC with the following conditions (Column: Lux 5 um Cellulose-4 2.12*25 cm, 5 μm; Mobile Phase A: Hex(0.3% IPAmine)—HPLC, Mobile Phase B: EtOH—HPLC; Flow rate: 20 mL/min; Gradient: isocratic 50; Wave Length: 220/254 nm; Frontpeak (min): 7.689; Postpeak (min): 10.526; Sample Solvent: EtOH—HPLC; Injection Volume: 0.5 mL, Number Of Runs: 36) to afford Frontpeak:Compound VIIIA (347.7 mg, 12.24%) as a white solid and Postpeak: Compound VIIIB (512.3 mg, 18.69%) as a white solid.

Compound VIIIA: ¹H NMR (400 MHz, DMSO-d6) δ 10.72 (s, 1H), 7.35-7.13 (m, 5H), 6.76 (dd, J=10.3, 1.9 Hz, 1H), 6.63 (dd, J=19.7, 10.4 Hz, 1H), 6.00 (d, J=7.3 Hz, 1H), 5.15 (d, J=4.1 Hz, 1H), 4.67 (t, J=5.7 Hz, 1H), 4.49 (dd, J=19.2, 5.5 Hz, 1H), 4.25 (dd, J=8.1, 4.8 Hz, 1H), 4.13-4.02 (m, 2H), 3.60 (dd, J=7.9, 5.8 Hz, 1H), 2.93 (dt, J=11.0, 6.1 Hz, 2H), 2.66 (dd, J=13.4, 7.9 Hz, 1H), 2.34-2.26 (m, 1H), 2.24 (s, 1H), 2.13 (t, J=9.8 Hz, 2H), 1.69 (d, J=12.2 Hz, 1H), 1.59 (t, J=11.5 Hz, 1H), 1.47 (d, J=16.0 Hz, 1H), 1.43 (s, 3H), 1.32 (d, J=14.6 Hz, 2H), 1.17-1.02 (m, 1H), 0.85 (s, 3H), 0.79 (d, J=7.2 Hz, 3H). MS (ESI)(m/z) [M+H]⁺ calculated: 554.3; found: 554.3.

Compound VIIIB: ¹H NMR (400 MHz, DMSO-d6) δ 10.66 (s, 1H), 7.34-7.14 (m, 5H), 6.50-6.35 (m, 2H), 6.27 (dd, J=10.1, 1.8 Hz, 1H), 5.21-5.11 (m, 1H), 4.94 (d, J=2.7 Hz, 1H), 4.68 (d, J=7.1 Hz, 1H), 4.50 (dd, J=19.2, 5.2 Hz, 1H), 4.17-4.02 (m, 2H), 3.62 (dd, J=7.8, 5.9 Hz, 1H), 2.94 (ddt, J=13.5, 8.5, 4.4 Hz, 2H), 2.73-2.52 (m, 2H), 2.33-2.08 (m, 4H), 1.74 (d, J=13.1 Hz, 1H), 1.61 (q, J=11.7 Hz, 1H), 1.47-1.38 (m, 4H), 1.33 (d, J=11.7 Hz, 1H), 1.10-1.02 (m, 1H), 0.86 (s, 3H), 0.79 (d, J=7.2 Hz, 3H). MS (ESI)(m/z) [M+H]⁺ calculated: 554.3; found: 554.3.

### Synthesis of Compounds IXA and IXB

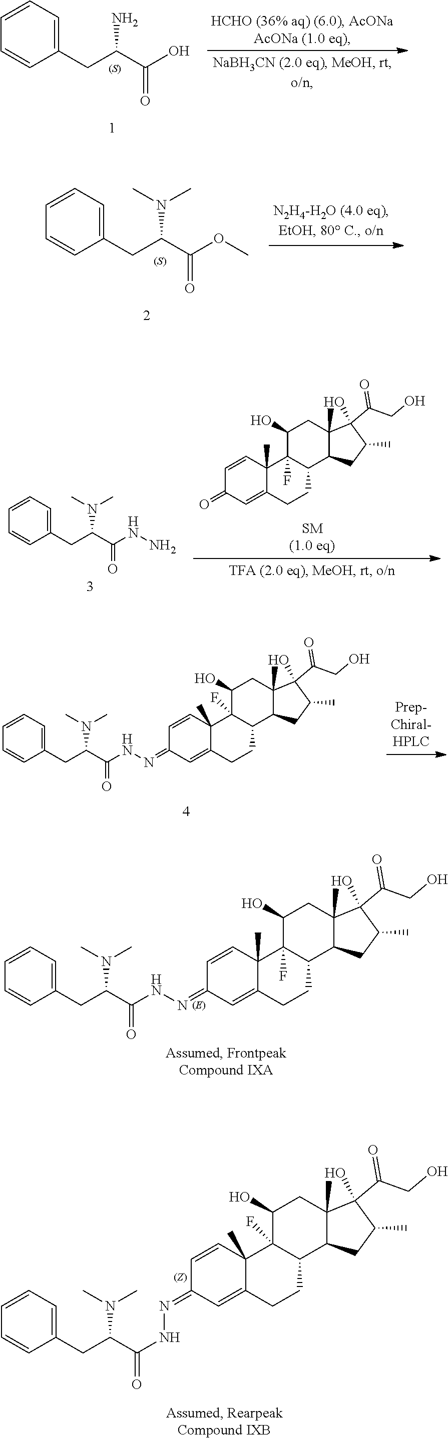

To a stirred solution of compound 1 (3.0 g, 16.73 mmol, 1.0 equiv) and HCHO (10.22 mL, 100.43 mmol, 6 equiv, 36%) in MeOH (15 mL) were added AcONa (1.37 g, 16.73 mmol, 1.0 equiv) and NaBH3CN (2.10 g, 33.47 mmol, 2.0 equiv) at 0° C. The resulting mixture was stirred overnight at room temperature. The resulting mixture was diluted with water (30 mL). The resulting mixture was extracted with EtOAc (2×50 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (5:1) to afford compound 2 (1.6 g, 46.11%) as a white solid. MS (ESI)(m/z): [M+1]⁺ calculated: 208.2; found: 208.3.

To a stirred solution of compound 2 (1.6 g, 7.71 mmol, 1 equiv) and hydrazine hydrate (1.55 g, 30.87 mmol, 4 equiv) in EtOH (10 mL) at room temperature. The resulting mixture was stirred overnight at 80° C. The resulting mixture was concentrated under vacuum. The residue was purified by reversed-phase flash chromatography with the following conditions: column, C18 silica gel; mobile phase, MeCN in Water (10 mmol/L NH4HCO3), 10% to 50% gradient in 10 min; detector, UV 254 nm. This resulted in compound 3 (0.8 g, 50.00%) as a light yellow oil. MS (ESI)(m/z): [M+1]⁺ calculated: 208.1; found: 208.1.

To a stirred solution of compound 3 (400 mg, 1.93 mmol, 1 equiv) and dexamethasone (757.38 mg, 1.93 mmol, 1.00 equiv) in MeOH (10.0 mL) was added TFA (0.29 mL, 3.86 mmol, 2.0 equiv) at room temperature. The resulting mixture was stirred overnight at room temperature. The resulting mixture was concentrated under vacuum. The crude product (410 mg) was purified by Chiral-Prep-HPLC with the following conditions (2 #SHIMADZU (HPLC-01)): Column, YMC-Actus Triart C₁₈ ExRS, 30*150 mm, 5 μm; mobile phase, 10 mmol NH4HCO3+0.05% NH3H2O and ACN (35% ACN up to 65% in 8 min); Detector, UV 254 nm to afford compound 4(200 mg, 17.10%) as a white solid. MS (ESI)(m/z): [M+1]⁺ calculated: 582.3; found: 582.3.

The compound 4 was purified by Chiral-Prep-HPLC with the following conditions (Column: CHIRALPAK ID, 2*25 cm, 5 μm; Mobile Phase A: Hex(0.1% FA)—HPLC, Mobile Phase B: IPA—HPLC; Flow rate: 20 mL/min; Gradient: 40% B to 40% B in 33 min; Wave Length: 220/254 nm; Frontpeak (min): 11.911; Postpeak (min): 25.866; Sample Solvent: IPA—HPLC; Injection Volume: 1 mL; Number Of Runs: 5) to afford Frontpeak: Compound IXA (23.2 mg, 10.73%) as a white solid and Postpeak: Compound IXB (15.0 mg, 6.84%) as a white solid.

Compound IXA: 1H NMR (400 MHz, Methanol-d4) δ 7.33-7.16 (m, 5H), 6.70 (d, J=10.4 Hz, 1H), 6.26 (dd, J=10.4, 1.9 Hz, 1H), 6.10 (s, 1H), 4.61 (dd, J=19.3, 6.1 Hz, 1H), 4.32-4.13 (m, 2H), 3.54 (s, 1H), 3.20-3.03 (m, 3H), 2.81-2.69 (m, 1H), 2.68-2.53 (m, 7H), 2.48-2.31 (m, 1H), 2.29 (s, 2H), 2.26 (s, 1H), 2.21 (td, J=11.6, 8.0 Hz, 1H), 1.83-1.65 (m, 2H), 1.50 (s, 3H), 1.44 (dd, J=13.7, 2.2 Hz, 1H), 1.20 (ddd, J=12.1, 8.1, 4.0 Hz, 1H), 1.00 (d, J=3.5 Hz, 3H), 0.88 (t, J=7.4 Hz, 3H). MS (ESI)(m/z) [M+H]⁺ calculated: 582.3; found: 582.3.

Compound IXB: 1H NMR (400 MHz, Methanol-d4) δ 7.33-7.22 (m, 4H), 7.26-7.15 (m, 1H), 6.60 (d, J=10.1 Hz, 1H), 6.35 (dd, J=10.1, 2.1 Hz, 1H), 6.04 (s, 1H), 4.64 (s, 1H), 4.29 (s, 1H), 4.22 (d, J=10.2 Hz, 1H), 3.55-3.47 (m, 1H), 3.19-3.01 (m, 3H), 2.76-2.60 (m, 1H), 2.54 (d, J=1.4 Hz, 6H), 2.40-2.28 (m, 1H), 2.30-2.24 (m, 1H), 2.22 (dd, J=13.6, 5.7 Hz, 2H), 1.88-1.74 (m, 1H), 1.71 (d, J=11.6 Hz, 1H), 1.49 (s, 3H), 1.57-1.41 (m, 1H), 1.31 (s, 1H), 1.22 (ddd, J=12.1, 8.1, 4.1 Hz, 1H), 1.00 (s, 3H), 0.89 (d, J=7.2 Hz, 3H). MS (ESI)(m/z) [M+H]⁺ calculated: 582.3; found: 582.3.

### Example 9: Synthesis of Compound X

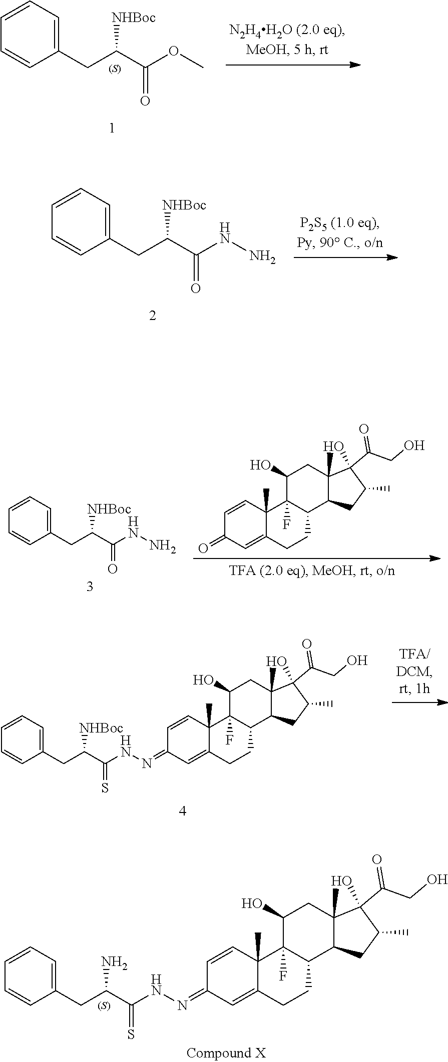

To a stirred solution of compound 1(100 mg, 0.35 mmol, 1 equiv) in MeOH (20 mL) was added hydrazine hydrate (0.87 mL, 14.32 mmol, 2.0 equiv, 80%) at 0° C. The resulting mixture was stirred for 5 h at room temperature. The resulting mixture was diluted with water (20 mL). The resulting mixture was extracted with EtOAc (2×50 mL). The combined organic layers were washed with brine (2×100 mL), dried over anhydrous Na2SO4. After filtration, the filtrate was concentrated under reduced pressure. This resulted in compound 2 (1.5 g, 75.00%) as a white solid. The crude product was used in the next step directly without further purification. MS (ESI)(m/z): [M+1]⁺ calculated: 280.2; found: 280.3.

To a stirred solution of compound 2 (500 mg, 1.79 mmol, 1 equiv) in Pyridine (8.0 mL) were added P2S5 (397.81 mg, 1.790 mmol, 1.00 equiv) at room temperature. The resulting mixture was stirred overnight at 90° C. The resulting mixture was diluted with water (50 mL). The resulting mixture was extracted with EtOAc (2×50 mL). The combined organic layers were washed with brine (2×100 mL), dried over anhydrous Na2SO4. After filtration, the filtrate was concentrated under reduced pressure. This resulted in compound 3(320 mg, 60.52%) as a yellow solid. The crude product was used in the next step directly without further purification. MS (ESI)(m/z): [M+1]⁺ calculated: 296.2; found: 296.2.

To a stirred solution of compound 3 (320 mg, 1.08 mmol, 1.0 equiv) and dexamethasone (425.15 mg, 1.08 mmol, 1.0 equiv) in MeOH (5.0 mL) were added TFA (0.16 mL, 2.16 mmol, 2.0 equiv) at room temperature. The resulting mixture was stirred overnight at room temperature. The resulting mixture was concentrated under reduced pressure. The residue was purified by silica gel column chromatography, eluted with PE/EA (2:1) to afford compound 4 (200 mg, 27.56%) as a yellow solid. MS (ESI)(m/z): [M+1]⁺ calculated: 670.3; found: 670.4.

To a stirred solution of compound 4 (200 mg, 0.30 mmol, 1 equiv) in DCM (3.0 mL) was added TFA (1.0 mL) at room temperature. The resulting mixture was stirred for 1 h at room temperature. The resulting mixture was concentrated under reduced pressure. The residue was purified by reversed-phase flash chromatography with the following conditions: column, C18 silica gel; mobile phase, MeCN in Water (10 mmol/L NH4HCO3), 30% to 50% gradient in 30 min; detector, UV 254 nm. to afford Compound X (27.9 mg, 16.40%) as a yellow solid.

Compound X: 1H NMR (400 MHz, Methanol-d4) δ 7.41-7.28 (m, 6H), 6.93 (d, J=10.0 Hz, 1H), 6.71 (d, J=3.8 Hz, 1H), 4.64-4.59 (d, J=14.3 Hz, 1H), 4.32-4.22 (m, 3H), 3.39 (dd, J=13.2, 7.2 Hz, 1H), 3.24-3.10 (m, 2H), 2.79 (td, J=13.7, 6.2 Hz, 1H), 2.52 (dtd, J=29.0, 11.9, 4.8 Hz, 2H), 2.27 (dd, J=25.3, 11.9 Hz, 2H), 2.01-1.92 (m, 1H), 1.81-1.75 (d, J=11.9 Hz, 1H), 1.61-1.48 (m, 5H), 1.22 (dt, J=14.9, 7.2 Hz, 1H), 1.03 (s, 3H), 0.88 (dd, J=7.4, 2.5 Hz, 3H). MS (ESI)(m/z) [M+H]⁺ calculated: 570.3; found:570.1.

### Example 10 Synthesis of Compound XI

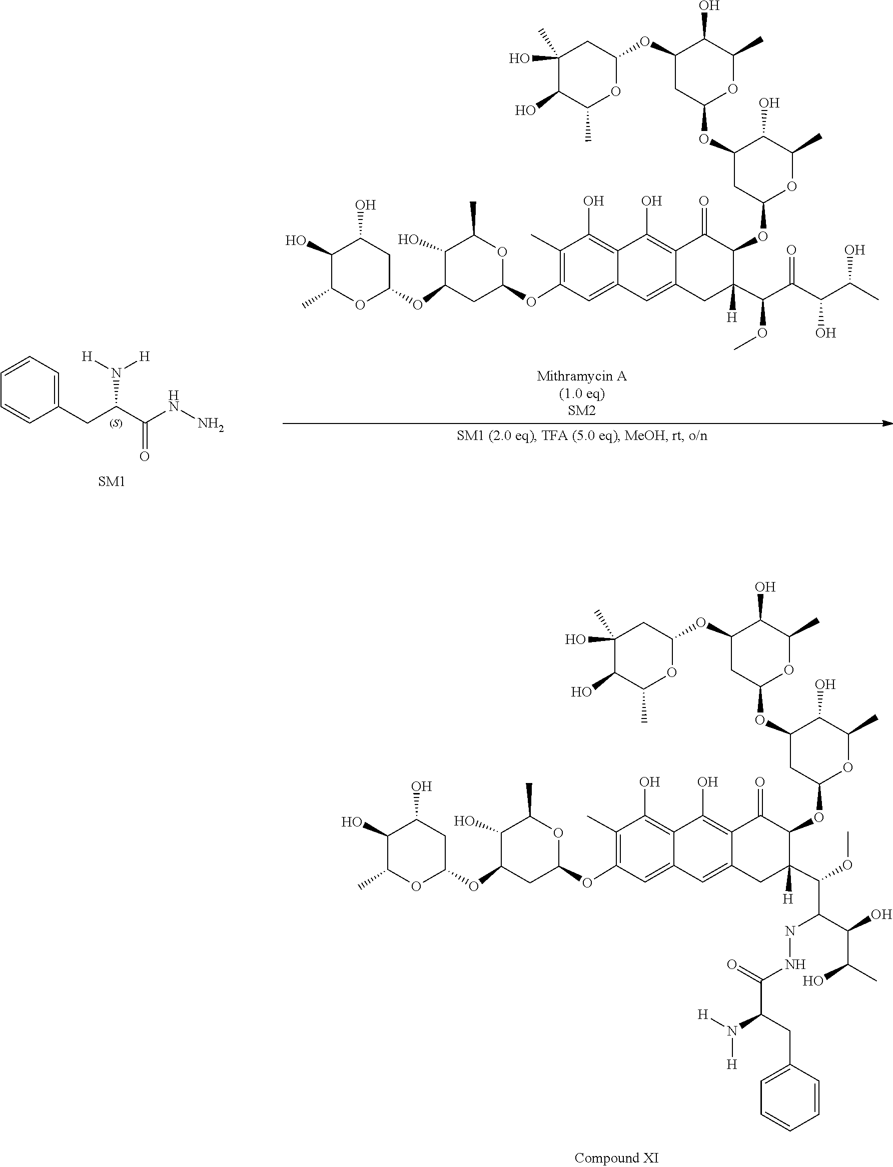

Into an 8 mL vial were added Mithramycin A (1 equiv), MeOH, hydrazine (2 equiv) and TFA (2 equiv) at room temperature. The resulting mixture was stirred overnight at room temperature. The resulting mixture was concentrated under reduced pressure. The crude product was purified by Prep-HPLC to afford Compound XI.

Compound XI: ¹H NMR (400 MHz, DMSO-d6) δ 11.09 (s, 1H), 7.34 (s, 1H), 7.31 (d, J=6.4 Hz, 4H), 7.25 (t, J=6.7 Hz, 2H), 6.33 (s, 1H), 6.24 (s, 1H), 6.17 (s, 1H), 5.96 (s, 1H), 5.23 (s, 1H), 5.17 (d, J=9.4 Hz, 1H), 4.96 (s, 2H), 4.87 (dd, J=13.8, 8.2 Hz, 5H), 4.61 (td, J=24.0, 20.6, 11.9 Hz, 5H), 4.22-4.13 (m, 4H), 4.03 (s, 1H), 3.96 (s, 2H), 3.75 (d, J=11.8 Hz, 3H), 3.62 (s, 1H), 3.55 (s, 1H), 3.24 (s, 5H), 2.95 (d, J=9.3 Hz, 2H), 2.88 (s, 1H), 2.77 (t, J=8.6 Hz, 5H), 2.40 (s, 2H), 2.28 (d, J=8.7 Hz, 1H), 2.04-1.90 (m, 5H), 1.76 (d, J=12.5 Hz, 2H), 1.67-1.57 (m, 2H), 1.50-1.33 (m, 3H), 1.23 (d, J=7.1 Hz, 5H), 1.21-1.06 (m, 23H). MS (ESI)(m/z) [M+H]⁺ calculated: 1246.6; found: 1246.6.

### Example 11 Synthesis of Compound XII

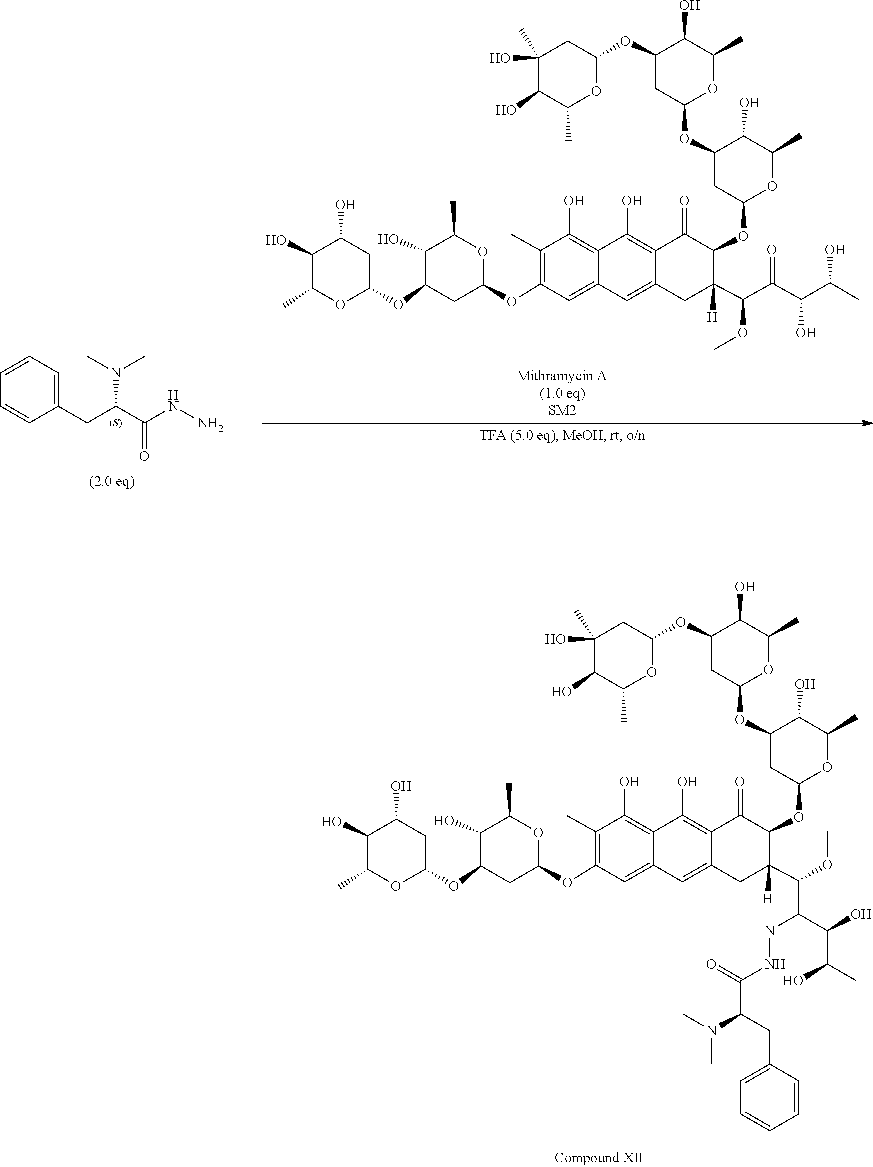

Into an 8 mL vial were added Mithramycin A (1 equiv), MeOH, hydrazine (2 equiv) and TFA (2 equiv) at room temperature. The resulting mixture was stirred overnight at room temperature. The resulting mixture was concentrated under reduced pressure. The crude product was purified by Prep-HPLC to afford Compound XII.

Compound XII: ¹H NMR (300 MHz, DMSO-d6) δ 14.80 (d, J=12.4 Hz, 1H), 10.72 (s, 1H), 7.35 (d, J=7.5 Hz, 2H), 7.25 (t, J=6.6 Hz, 5H), 6.61 (s, 1H), 6.38 (s, 1H), 6.22 (s, 1H), 6.15 (s, 2H), 5.24 (d, J=9.3 Hz, 1H), 5.06 (s, 1H), 5.03-4.86 (m, 10H), 4.69 (d, J=6.7 Hz, 1H), 4.42 (s, 1H), 4.26 (s, 2H), 4.16 (s, 1H), 4.04 (d, J=5.5 Hz, 2H), 3.83 (s, 3H), 3.70 (s, 1H), 3.61 (s, 2H), 3.48 (s, 1H), 3.23 (d, J=16.7 Hz, 1H), 2.94 (s, 3H), 2.79 (d, J=10.4 Hz, 6H), 2.39 (s, 3H), 2.29 (d, J=8.1 Hz, 11H), 2.08 (d, J=6.6 Hz, 4H), 1.98 (s, 6H), 1.87 (s, 4H), 1.76 (d, J=13.1 Hz, 1H), 1.49-1.36 (m, 3H), 1.21 (s, 13H), 1.30-1.11 (m, 22H), 1.04 (s, 9H), 1.04 (d, J=16.4 Hz, 2H), 0.84 (d, J=6.4 Hz, 1H). MS (ESI)(m/z) [M+H]⁺ calculated: 1274.6; found: 1274.6.

### In-Vitro and In-Vivo Biological Studies

Synthesis of different compounds was followed by testing each compound in different preclinical models for lead optimization. Initial tests included i) chemical stability at two different pH (4.5 and 7.4), ii) human and rodent plasma stability iii) rat liver microsomal stability, iv) cellular permeability v) pharmacokinetic and tissue distribution analysis and vi) biological assays such as glucocorticoid receptor (GR) binding were performed.

In vivo PK studies were conducted in accordance with the guidelines of Institutional Animal Care and Use Committee (IACUC). No abnormal clinical symptoms were observed in rats during the entire experiment.

### Chemical Stability

Chemical stability of the prodrug compounds was tested at pH 4.5 and pH 7.4 (pH adjusted in phosphate buffer saline). Both buffer solutions at two pHs were prepared in house. 2 μL of 500 μM stock solution of the prodrug was added to each vial containing 198 μL PBS at pH 7.4 or pH 4.5 and mixed evenly. The assay was performed in duplicate. The final concentration of the test compound was 5 μM. samples were incubated at 37° C. at 600 rpm. The initiation of the reaction was staggered so all the time points (0, 30, 60, 120, 180 and 240 minutes) are terminated at the same time with 1000 L cold quench solution (acetonitrile containing internal standards (IS, 200 nM Labetalol, 100 nM Alprazolam and 2 μM Ketoprofen)). Samples from individual vials were used for the different time points. The samples were vortexed for 2 minutes and centrifuged at 3,220 g for 5 minutes at 4° C. 100 L of the supernatant was transferred to a new plate. The supernatant was diluted with 100 L or 200 L water according to the LC/MS signal response and peak shape. The samples were mixed well and analyzed using LC/MS/MS. The remaining percentage of the parent drug versus reaction time was used to calculate the t1/2 value.

### Plasma Stability

Rat and human plasma were obtained from qualified vendors. 5 μL of 500 M of the test article solution was added to 495 μL of pre-incubated human or rat plasma to reach a final concentration of 5 μM. The final concentration of organic solvents was not more than 0.5%. The assay was performed in duplicate. The reaction samples were incubated in a 37° C. water bath with shaking at approximately 60 rpm. 50 μL of the reaction samples were collected at 0, 30, 60, 120 and 180 minutes. The reaction was stopped by adding 300 μL of room temperature quench solution (acetonitrile containing internal standards (IS, 200 nM labetalol, 100 nM alprazolam and 2 μM ketoprofen)). The samples were vortexed for 5 minutes followed by centrifugation of the samples at 3,220 g for 30 minutes at room temperature to precipitate the protein. 100 L of the supernatant was transferred to a new plate and diluted with 100 L or 200 L water according to the LC/MS signal response and peak shape. The samples were mixed well and analyzed using LC/MS/MS. The remaining percentage of parent drug versus reaction time was used to calculate the t1/2 value.

### Metabolic Stability:

Rat liver microsomes were obtained from qualified vendors and metabolic stability of the test compound was determined at 1 uM. The compound was incubated at 37° C. with 0.5 mg microsome/mL in the buffer in the presence of 1 mM NADPH. Aliquots of 50 μL were taken from the reaction solution at 0, 15, 30, 45 and 60 minutes. The reaction was stopped by the addition of 4 volumes of cold acetonitrile with internal standards (IS, 200 nM labetalol, 100 nM alprazolam and 2 μM ketoprofen). Precipitated proteins were removed by centrifugation, followed by withdrawing and diluting the supernatant for analysis. Two positive controls and negative controls (without NADPH) were used in the study. All samples were analyzed in duplicates. LC/MS/MS was performed for detection and quantification of the test compounds. Linear regression was determined using the remaining percentage of the parent drug at each incubation time and t1/2 value was calculated.

### Permeability Study Using Caco-2 Cell Monolayers:

The objective of this study was to evaluate the bidirectional permeability and absorption mechanisms of the test compounds. CaCO-2 cells were cultured, and a monolayer was developed in-house in 96 well-plates over 18 days using standard protocol. The monolayer was tested for the integrity of the cell monolayer before using.

Test compounds were added to the transwell insert (apical compartment) to determine the rate of drug transport in the apical to basolateral direction. To determine the rate of drug transport in the basolateral to apical direction, the test compound was added to the receiver plate wells (basolateral compartment). Appropriate amount of HBSS (10 mM HEPES, pH 7.4) was added in both compartments for a final volume of 200 μL. The plates were incubated at 37° C. with shaking at 150 rpm on a rotary shaker for 2 hours. At the end of the transport period, 50 μL from the apical and basolateral wells were collected and 4 volume of cold acetonitrile containing internal standards (IS: 100 nM alprazolam, 200 nM labetalol, and 2 μM ketoprofen) was added to each sample to terminate the reaction. The samples were vortexed and centrifuged at 3,800 g for 20 minutes. An aliquot of 150 μL of the supernatant was used for LC/MS/MS analysis. All samples were analyzed in duplicates. Samples were analyzed through LC/MS/MS for estimating the permeability and efflux of the compounds across Caco-2 cell monolayers.

### Glucocorticoid Receptor Binding Assay

Glucocorticoid receptor (GR) binding of prodrugs was determined using TR-FRET GR competitive binding assay. Three-point (0.1, 1 and 10 μM) binding was evaluated for each prodrug. Dexamethasone was used as the positive control.

### Pharmacokinetic Studies in Rats:

Six to eight weeks old Sprague-Dawley rats were procured from a qualified provider. Test compounds were introduced in rats by a single intravenous administration. A liquid formulation was made (2 mg/kg dexamethasone or equivalent prodrug) in 20% HP-β-CD and administered via the tail vein to the rats which were subject to overnight fasting as a prerequisite. Blood and brain samples were collected at three timepoints (15, 60 and 360 min) from three rats (3 replicates) after dosing. Blood samples (0.1, 0.2 or 0.5 mL) were collected with a 1 mL syringe containing anticoagulants K2EDTA. Blood samples were centrifuged to obtain plasma for analysis. Additional blood samples were collected at 15, 30, 60, 120, 240 min from the 360 min group. After collection at each time point, brain samples were frozen at −75° C. Before analysis, a portion of the brain sample was weighed and homogenized with phosphate buffer saline. Concentrations of the prodrug and dexamethasone released from the prodrug, in both plasma and brain tissue, were measured by LC/MS/MS. Data analysis and pharmacokinetic parameters were generated using WinNonlin software.

Table 1 shows the chemical stability at two different pH (4.5 and 7.4) for prodrug compounds according to the embodiments of the present invention. As shown in Table 2, the prodrug compounds were stable even at pH 7.4.

TABLE 1

| | Chemical Stability, | Chemical Stability, |
|---|---|---|
| Compound | pH 4.5 | pH 7.4 |
| Compound I | Yes | Yes |
| Compound II | Yes | Yes |
| Compound IIIA | Yes | Yes |
| Compound IIIB | Yes | Yes |
| Compound IVA | Yes | Yes |
| Compound IVB | Yes | Yes |
| Compound VA | Yes | Yes |
| Compound VB | Yes | Yes |
| Compound VIIA | Yes | Yes |
| Compound VIIB | Yes | Yes |
| Compound VIII | Yes | Yes |
| Compound IXA | NA | NA |
| Compound IXB | NA | NA |

Table 2 shows human plasma stability and rat plasma stability for prodrug compounds according to embodiments of the present invention. As shown in Table 2, the prodrug compounds showed human plasma stability.

TABLE 2
Human plasma stability and rat plasma stability

| | Human Plasma Stability | Rat Plasma Stability |
|---|---|---|
| Compound | t½ min | t½ min |
| Compound I | >240 | NA |
| Compound II | >240 | NA |
| Compound IIIA | >240 | >240 |
| Compound IIIB | >240 | NA |
| Compound IVA | >240 | >240 |
| Compound IVB | >240 | NA |
| Compound VA | NA | >240 |
| Compound VB | NA | NA |
| Compound VIIA | NA | >240 |
| Compound VIIB | NA | >240 |
| Compound VIIA | NA | >240 |
| Compound VIIIB | NA | >240 |
| Compound IXA | NA | >240 |
| Compound IXB | NA | >240 |
| Compound X | NA | 125 |
| Compound XI | NA | >240 |
| Compound XII | NA | >240 |

Note:
t½ > 240 min was considered to be stable

Table 3 shows rat liver microsomal stability data for prodrug compounds

TABLE 3
Rat liver microsomal stability

| | RLM |
|---|---|
| Compound | t½ min |
| Compound IIIA | 33 |
| Compound IIIB | 81 |
| Compound IVA | 35 |
| Compound IVB | 108 |
| Compound VA | 44 |

Table 4 shows peremeability data for prodrug compounds

TABLE 4
Permeability data (CaCo/MCDK2)

| Compound | Permeability | |
|---|---|---|
| Compound IIIB | A-B = 1.13 × 10⁻⁶, cm/s | |
| (MDCK) | B-A = 3.50 × 10⁻⁶, cm/s | |
| | Efflux ratio: 3.03 | |
| Compound IVA | A-B = 0.2 × 10⁻⁶, cm/s | |
| (Caco2) | B-A = 4 × 10⁻⁶, cm/s | |
| | Efflux ratio: 20 | |
| Compound IV | A-B = 1.21 × 10⁻⁶, cm/s | |
| (MDCK) | B-A = 1.73 × 10⁻⁶, cm/s | |
| | Efflux ratio: 1.44 | |
| Compound VIIA | A-B = 0.17 × 10⁻⁶, cm/s | A-B = 0.15 × 10⁻⁶, cm/s |
| CaCO2/MDCK | B-A = 11 × 10⁻⁶, cm/s | B-A = 1.29 × 10⁻⁶, cm/s |
| | Efflux ratio: 65 | Efflux ratio: 8.48 |
| Compound VIIB | A-B = 0.13 × 10⁻⁶, cm/s | A-B = 0.12 × 10⁻⁶, cm/s |
| CaCO2/MDCK | B-A = 9 × 10⁻⁶, cm/s | B-A = 0.87 × 10⁻⁶, cm/s |
| | Efflux ratio: 68 | Efflux ratio: 7.14 |

| | |
|---|---|
| Compound VIIIA | A-B = 0.44 × 10⁻⁶, cm/s |
| (MDCK) | B-A = 1.12 × 10⁻⁶, cm/s |
| | Efflux ratio: 2.54 |
| Compound VIIIB | A-B = 0.47 × 10⁻⁶, cm/s |
| (MDCK) | B-A = 1.04 × 10⁻⁶, cm/s |
| | Efflux ratio: 2.22 |
| Compound X | A-B = 0.33 × 10⁻⁶, cm/s |
| (Caco2) | B-A = 4 × 10⁻⁶, cm/s |
| | Efflux ratio: 12 |
| Compound X | A-B = <0.17 × 10⁻⁶, cm/s |
| (MDCK) | B-A = 1.82 × 10⁻⁶, cm/s |
| | Efflux ratio: >10.69 |

Table 5 shows glucocorticoid receptor (GR) binding data for prodrug compounds according to embodiments of the present invention. As shown in Table 3 the prodrugs of the present invention showed significantly lower GR binding as compared to dexamethasone.

TABLE 5
Glucocorticoid receptor (GR) binding data for prodrugs compounds

| Compound | GR Binding Data |
|---|---|
| Dexamethasone | 93% @100 nM, 50% @ 10 nM, 16% @ 1 nM |
| Compound IIIB | 68% @ 10 uM, 8% @ 1 uM, 8% @ 0.1 uM |
| Compound IVA | 64% @ 10 uM, 8% @ 1 uM, 4% @ 0.1 uM |
| Compound IVB | 72% @ 10 uM, 16% @ 1 uM, 3% @ 0.1 uM |
| Compound VIIIB | 72% @ 10 uM, 26% @ 1 uM, 19% @ 0.1 uM |

Table 6 shows in-vitro biological assay data a mixture of prodrug isomers according to embodiments of the present description.

TABLE 6
In-vitro biological assay data for a
mixture of prodrugs compounds VIA and VIB

| Chemical Stability | Stable |
|---|---|
| Human Plasma Stability | >240 |
| t½ min | |
| Rat Plasma Stability t½ min | >240 |
| Human liver microsomal stability | 34 |
| t½ min | |
| Rat liver microsomal stability | 43 |
| t½ min | |
| MDCK | A-B = 1.1 × 10⁻⁶, cm/s |
| | B-A = 3.9 × 10⁻⁶, cm/s |
| GR Binding Data | 64% @ 10 uM, |
| | 17% @ 1 uM, |
| | 3% @ 0.1 uM |

### In Vivo Pharmacokinetics and Tissue Distribution:

A mixture of compounds VIA and VIB were tested in rats and the concentration of the prodrugs and active molecules (dexamethasone) were measured in the plasma and brains at three different time points. Table 7 shows that the prodrug compounds exhibited higher concentrations of dexamethasone in the brain relative to the plasma.

TABLE 7
In-vivo analysis of Dexamethasone, a mixture of Compounds VI and
VB, and Dexamethasone released from the prodrugs in rat

| | Time | | Compounds |
|---|---|---|---|
| | (min) | Dexamethasone | VI and VIB |
| Conc of Prodrug in plasma | 15 | NA | 267 ± 39.7 |
| | 60 | NA | 97.6 ± 21.9 |
| | 240 | NA | NA |
| | 360 | NA | NA |
| | 540 | NA | NA |
| | 1440 | NA | NA |
| Conc in Prodrug in Brain | 15 | NA | 10.8 ± 3.03 |
| | 60 | NA | 7.34 |
| | 240 | NA | NA |
| | 360 | NA | NA |
| | 540 | NA | NA |
| | 1440 | NA | NA |
| Brain/Plasma % of | 15 | NA | 0.04 |
| Prodrug | 60 | NA | 0.08 |
| | 240 | NA | NA |
| | 360 | NA | NA |
| | 540 | NA | NA |
| | 1440 | NA | NA |
| Conc of Dex in plasma | 15 | 1400 ± 731 | 6.60 ± 4.74 |
| | 60 | 1247 ± 28 | 13.7 ± 3.60 |
| | 240 | NA | NA |
| | 360 | 250 ± 19 | 22.6 ± 9.16 |
| | 540 | NA | 10.0 ± 2.17 |
| | 1440 | NA | 15.9 ± 3.05 |
| Conc of Dex in Brain | 15 | 54.1 ± 17.2 | 25.6 ± 34.4 |
| | 60 | 84.1 ± 7.4 | 35.9 ± 4.41 |
| | 240 | NA | NA |
| | 360 | 42.9 ± 5.34 | 41.2 ± 22.9 |
| | 540 | NA | 23.8 ± 14.8 |
| | 1440 | NA | 38.9 ± 26.1 |
| Brain/Plasma % of Dex | 15 | 0.04 | 3.88 |
| | 60 | 0.07 | 2.62 |
| | 240 | NA | NA |
| | 360 | 0.17 | 1.82 |
| | 540 | NA | 2.39 |
| | 1440 | NA | 2.46 |

The concentration of dexamethasone in plasma released from the prodrugs was significantly lower than dexamethasone administered to the rats, while the concentration of dexamethasone in the brain was higher for the prodrugs. Thus, the data suggests that prodrugs compounds of the present invention have the potential to deliver adequate amounts of dexamethasone in the brain with less systemic exposure, thereby, reducing the toxicities of dexamethasone in the body.

While only certain features of several embodiments have been illustrated and described herein, many modifications and changes will occur to those skilled in the art. It is, therefore, to be understood that the appended claims are intended to cover all such modifications and changes as fall within the scope of the invention and the appended claims.

1. A compound having a formula (I) or a pharmaceutically acceptable salt thereof: 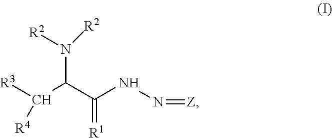
wherein Z is a therapeutic moiety;
R¹ is O or S;
R² is independently at each occurrence hydrogen or a C₁-C₃ alkyl group;
R³ is hydrogen or a C₁-C₃ alkyl group; and
R⁴ is a C₁-C₆ alkyl group, a C₆-C₁₀ aryl group, or a C₅-C₁₀ heteroaryl group.

2. The compound of claim 1, wherein the compound is an L and syn isomer of formula (I).

3. The compound of claim 1, wherein the compound is an L and anti isomer of formula (I).

4. The compound of claim 1, wherein the compound is a D and syn isomer of formula (I).

5. The compound of claim 1, wherein the compound is a D and anti isomer of formula (I).

6. The compound of claim 1, wherein the compound has a formula (II) or a pharmaceutically acceptable salt thereof:

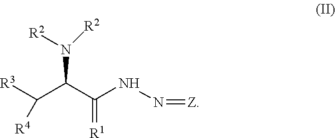

7. The compound of claim 1, wherein the compound has a formula (III) to (VI) or a pharmaceutically acceptable salt thereof:

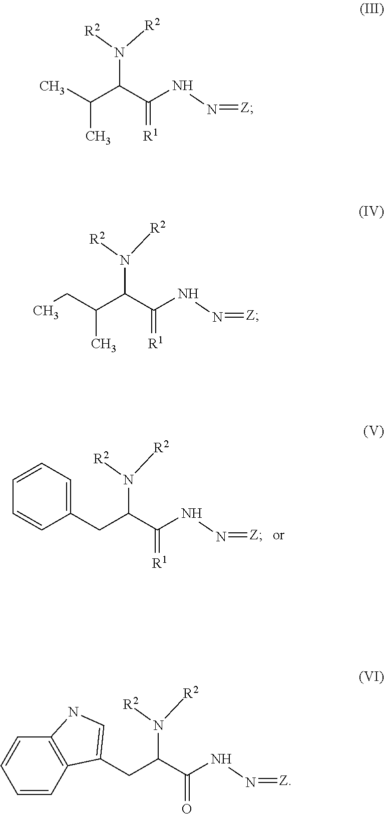

8. The compound of claim 1, when Z is a therapeutic moiety obtained from a therapeutic agent selected from the group consisting of an antibiotic, an anti-inflammatory agent, an anti-viral agent, an anti-cancer agent, an anti-infective agent, and combinations thereof.

9. The compound of claim 8, wherein Z is a therapeutic moiety obtained from a corticosteroid selected from the group consisting of dexamethasone, prednisone, prednisolone, triamcinolone, cortisone, hydrocortisone, and betamethasone.

10. The compound of claim 9, wherein Z is a dexamethasone residue.

11. The compound of claim 8, wherein Z is mithramycin residue.

12. A pharmaceutical composition comprising:
the compound of claim 1 or a pharmaceutically acceptable salt, solvate, hydrate, polymorph, or co-crystal thereof, and
a pharmaceutically carrier, diluent, or excipient.

13. A method of treating a brain disease comprising administering to a patient an effective amount of a pharmaceutical composition comprising a compound of formula (I) or a pharmaceutically acceptable salt thereof: 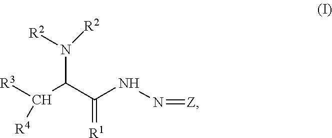
wherein Z is a therapeutic moiety;
R¹ is O or S;
R² is independently at each occurrence hydrogen or a C₁-C₃ alkyl group;
R³ is hydrogen or a C₁-C₃ alkyl group; and
R⁴ is a C₁-C₆ alkyl group, a C₆-C₁₀ aryl group, or a C₅-C₁₀ heteroaryl group.

14. The method of claim 13, wherein the compound is an L and syn isomer of formula (I).

15. The method of claim 13, wherein the compound is an L and anti isomer of formula (I).

16. The method of claim 13, wherein the compound is a D and anti isomer of formula (I).

17. The method of claim 13, wherein the compound is a D and syn isomer of formula (I).

18. The method of claim 13, wherein the compound has a formula (II) or a pharmaceutically acceptable salt thereof:

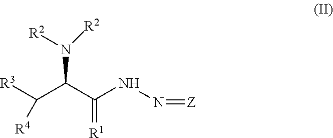

19. The method of claim 13, wherein the compound has a formula (III) to (VI) or a pharmaceutically acceptable salt thereof:

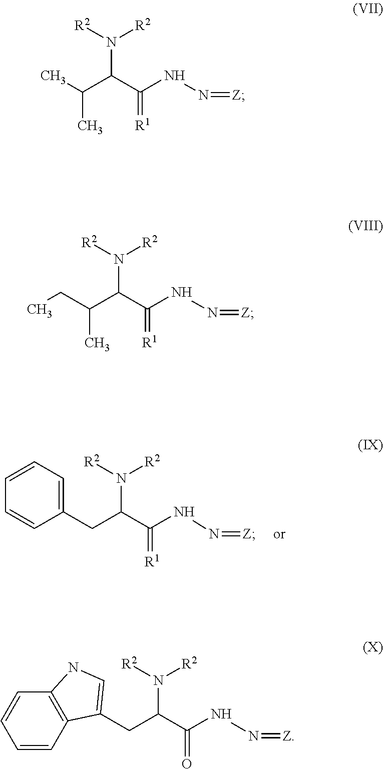

20. The method of claim 13, wherein the brain disease comprises glioblastoma, medulloblastoma, glioma, or a brain metastatic disease.
